(19) United States
(12) Patent Application Publication
(10) Pub. No.: US 2025/0057821 A1
(43) Pub. Date: Feb. 20, 2025

(54) METHODS OF TREATING MYELOID MALIGNANCIES USING BCL-2 INHIBITOR

(71) Applicant: BeiGene Switzerland GmbH, Basel (CH)

(72) Inventor: David Simpson, San Mateo, CA (US)

(73) Assignee: BeiGene Switzerland GmbH, Basel (CH)

(21) Appl. No.: 18/938,467

(22) Filed: Nov. 6, 2024

Related U.S. Application Data
(63) Continuation of application No. PCT/IB23/54902, filed on May 11, 2023.
(60) Provisional application No. 63/370,170, filed on Aug. 2, 2022.
(60) Provisional application No. 63/350,524, filed on Jun. 9, 2022.
(60) Provisional application No. 63/341,210, filed on May 12, 2022.

(51) Int. Cl. A61K 31/437; A61K 31/706; A61P 35/02

(52) U.S. Cl. CPC A61K 31/437; A61K 31/706; A61P 35/02

(57) ABSTRACT

The present disclosure provides methods of treating myeloid malignancies in a subject with a Bcl-2 inhibitor, in particularly 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide or a pharmaceutically acceptable salt thereof, in combination with azacitidine.

### CROSS-REFERENCE TO RELATED APPLICATIONS

This application is a continuation of International Application No. PCT/IB2023/054902, filed on May 11, 2023, which claims the benefit of U.S. Provisional Application No. 63/370,170, filed on Aug. 2, 2022, U.S. Provisional Application No. 63/350,524, filed on Jun. 9, 2022, and U.S. Provisional Application No. 63/341,210, filed on May 12, 2022. The disclosures of each of the aforementioned applications are incorporated herein by reference in their entireties.

### FIELD OF THE DISCLOSURE

Disclosed herein are methods of treating myeloid malignancies with a Bcl-2 inhibitor, in particularly 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide or a pharmaceutically acceptable salt thereof, in combination with azacitidine.

### BACKGROUND OF THE DISCLOSURE

Impaired apoptosis plays a central role in tumor development, tumor maintenance, and therapeutic resistance. Apoptosis can be triggered via two main pathways: the extrinsic or death-receptor-mediated pathway, and the intrinsic or mitochondrial pathway (Czabotar et al 2014). It is the intrinsic pathway that is more commonly perturbed in lymphoid malignancies. Cell death mediated through this pathway is regulated by members of a family of proteins related to B-cell lymphoma-2 (Bcl-2), which is considered to contain three subfamilies. The pro-survival subgroup (Bcl-2, Bcl-xL, Bcl-W, Mcl-1, A1/Bfl-1, and possibly Bcl-B) promotes cell survival by inhibiting their pro-apoptotic relatives. The pro-apoptotic BAX/BAK-like proteins, including BOK, are the essential effectors of apoptosis, and the BH3-only proteins (BIM, PUMA, BID, NOXA, BMF, BIK, and HRK) are the initiators of apoptosis (Anderson et al 2014). In healthy cells, the pro-survival Bcl-2 proteins bind and inhibit BAX and BAK after they have been partially activated, impairing the ability of BAX/BAK to oligomerize and form pores to induce mitochondrial outer membrane permeabilization. The BH3-only proteins are induced transcriptionally or post-transcriptionally in response to diverse stresses and initiate apoptosis by either binding the pro-survival Bcl-2 proteins, thereby unleashing BAX/BAK, or by directly activating these effectors of apoptosis. The various Bcl-2 family proteins have differential specificity of binding to one another, resulting in a complex but ordered network of interactions governing cell fate (Roberts 2016).

Bcl-2 was the first anti-apoptotic protein discovered in 1980s as a consequence of t(14;18) chromosomal translocation and the hallmark of FL. BCL-2 gene resides on chromosome 18q21.33. The Bcl-2 protein has 239 amino acids and a molecular weight of 26 kDa (Schenk et al 2017). Bcl-2 is widely expressed during development and becomes restricted upon maturation in many tissues (Kondo et al 2008). Mice lacking Bcl-2 succumb to polycystic kidney disease early in life because Bcl-2 is critical for the survival of renal epithelial progenitor cells during embryogenesis (Veis et al 1993). The Bcl-2-deficient mice also have abnormally reduced numbers of mature, resting B and T lymphocytes, and gray prematurely because of the aberrant death of melanocytes (Veis et al 1993, Yamamura et al 1996). Although originally believed to act as a classical growth-driving oncogene, it was later shown that Bcl-2 instead promotes malignant cell survival by attenuating apoptosis. Transgenic mice with pan-hematopoietic Bcl-2 expression (VavP-BCL-2) preferentially develop follicular lymphoma, preceded by florid germinal center hyperplasia (Egle et al 2004). Mice co-expressing BCL-2 and MYC transgenes developed lymphomas markedly faster than littermates expressing either transgene alone, validating BCL-2 as an oncogene (Adams and Cory 2007).

High Bcl-2 expression is almost universal in CLL, FL, MCL, and Waldenstrom macroglobulinemia (WM); in contrast, the levels of Bcl-2 expression are somewhat more variable among multiple myeloma (MM) and substantially more variable among DLBCL and B-lineage acute lymphoblastic leukemia (Roberts and Huang 2017). When Bcl-2 is overexpressed, the ratio of pro- and anti-apoptotic Bcl-2 family members is disturbed and apoptotic cell death can be prevented. Moreover, Bcl-2 protein is closely related to chemoresistance in hematological tumors. As Bcl-2-mediated resistance to intrinsic apoptosis is considered as a key to pathogenesis, targeting Bcl-2 can improve apoptosis and overcome drug resistance to cancer therapy. Thus, Bcl-2 has become an attractive target for therapeutic strategy in cancer.

Venetoclax (ABT-199) was approved for treating patients with chronic lymphocytic leukemia (CLL) and acute myeloid leukemia (AML). However, despite this high clinical activity and favorable safety profile, patients can develop acquired resistance to venetoclax over time with continuous treatment. Blombery et al demonstrated that the Gly101Val mutation (G101V mutation) in BCL-2 confers acquired refractoriness by reducing the binding affinity of venetoclax without disrupting the binding of pro-apoptotic proteins to Bcl-2. The novel Gly101Val mutation in Bcl-2 was identified at progression in 7 of 15 patients. This mutation is mainly found in patients after long-term exposure to venetoclax monotherapy (Tausch et at 2019).

WO2019/210828A disclosed a series of compounds having the following Formulas (III-B), (III-C), (III-D) or (III-E), or a stereoisomer thereof, or a pharmaceutically acceptable salt thereof, as Bcl-2 inhibitors,

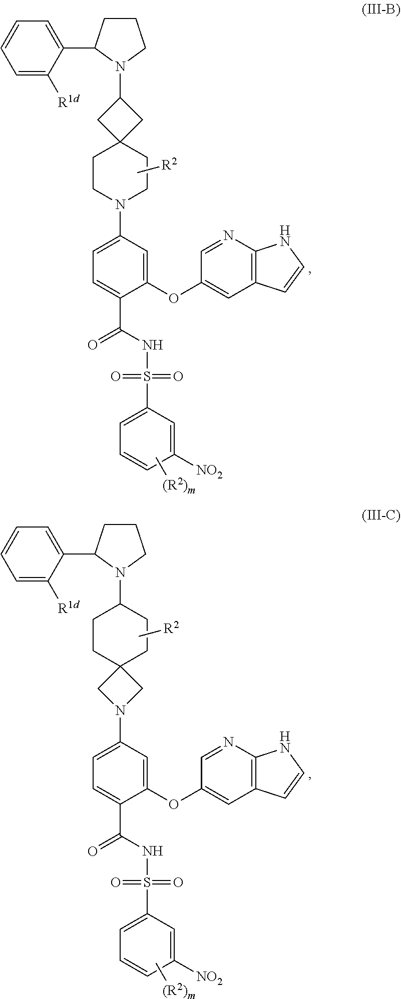

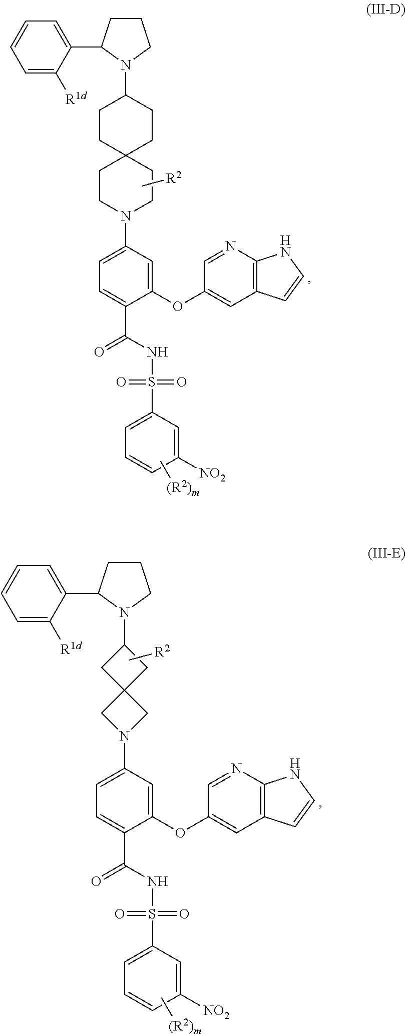

The compounds disclosed in WO2019/210828A are potent and selective Bcl-2 protein inhibitors.

### SUMMARY OF THE DISCLOSURE

The inventors of the present disclosure have found that a Bcl2 inhibitor having Formulas (III-B), (III-C), (III-D) or (III-E), in particularly 2-((1H-pyrrolo[2,3-b]pyridin-5-yl)oxy)-N-((4-((((1 r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide (Compound A) or a pharmaceutically acceptable salt thereof, in combination with azacitidine produced significant inhibition of tumor growth in acute myeloid leukemia (AML) as compared with the efficacy of each therapeutic as a single agent. Further, the combination therapy demonstrates significant inhibition of tumor growth in myeloid malignancies, including acute myeloid leukemia (AML) (either treatment-naîve [TN] unfit for intensive induction chemotherapy or relapsed/refractory [R/R]), myelodysplastic syndrome (MDS), or MDS/myeloproliferative neoplasm (MPN).

In a first aspect, disclosed herein is a method of treating myeloid malignancies with a Bcl-2 inhibitor in combination with azacitidine, wherein the Bcl-2 inhibitor is a compound represented by the following Formulas (III-B), (III-C), (III-D) or (III-E).

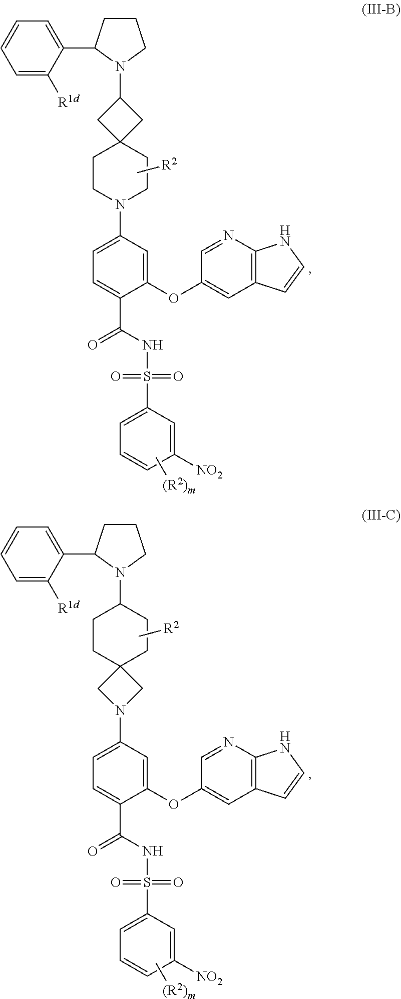

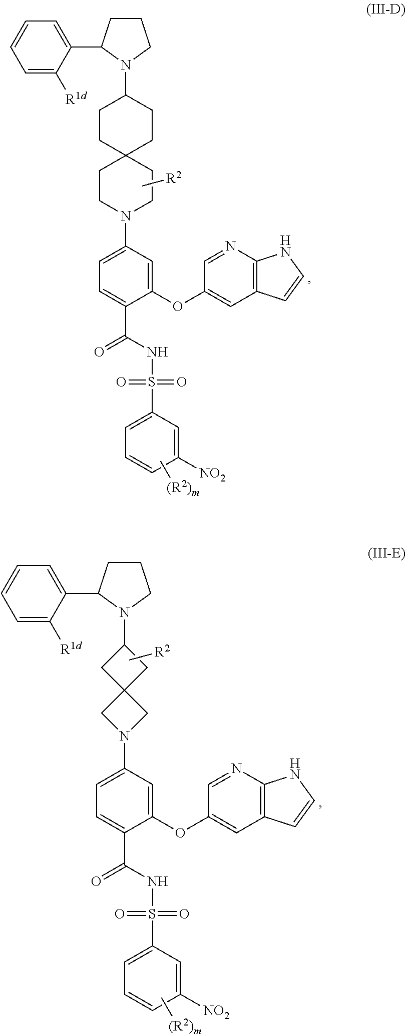

or a pharmaceutically acceptable salt thereof, or a stereoisomer thereof,
wherein,
R², at each occurrence, is independently selected from the group consisting of hydrogen, halogen, or —C₁₋₈alkyl optionally substituted with halogen;
R^{1d}, at each occurrence, is independently halogen, —C₁₋₈alkyl, —C₂₋₈alkenyl, —C₂₋₈alkynyl, cycloalkyl, heterocyclyl, aryl, heteroaryl, oxo, —CN, —NO₂, —OR^{Ba}, —SO₂R^{Ba}, —COR^{Ba}, —CO₂R^{Ba}, —CONR^{Ba}R^{Bb}, —C(═NR^{Ba})NR^{Bb}R^{Bc}, —NR^{Ba}R^{Bb}, —NR^{Ba}COR^{Bb}, —NR^{Ba}CONR^{Bb}R^{Bc}, —NR^{Ba}CO₂R^{Bb}, —NR^{Ba}SONR^{Bb}R^{Bc}, —NR^{Ba}SO₂NR^{Bb}R^{Bc}, or —NR^{Ba}SO₂R^{Bb}; wherein said —C₁₋₈ alkyl, —C₂₋₈alkenyl, —C₂₋₈alkynyl, cycloalkyl, heterocyclyl, aryl or heteroaryl are each independently optionally substituted with 1 to 4 substituents R^{Bd};
R^{Ba}, R^{Bb}, and R^{Bc}, are each independently hydrogen, —C₁₋₈alkyl, —C₂₋₈alkenyl, —C₂₋₈alkynyl, cycloalkyl, heterocyclyl, aryl, or heteroaryl, each of said —C₁₋₈alkyl, —C₂₋₈alkenyl, —C₂₋₈ alkynyl, cycloalkyl, heterocyclyl, aryl, or heteroaryl is optionally substituted with halogen, hydroxy, —NH₂ or —N(C₁₋₆alkyl)₂, —C₁₋₈alkyoxy, cycloalkyl, heterocyclyl, aryl, or heteroaryl;
R^{Bd}, at each occurrence, is independently hydrogen, halogen, oxo, —CN, —NO₂, —C₁₋₈alkyl, —C₂₋₈alkenyl, —C₂₋₈alkynyl, cycloalkyl, heterocyclyl, aryl, or heteroaryl, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, —C₁₋₈alkyoxy, cycloalkyl, heterocyclyl, aryl, or heteroaryl;
m is an integer of 1-4;
R⁵ is -L⁵-CyC,
   Wherein L⁵ is a direct bond, —(CR^{a}R^{b})ₜ—, —(CR^{a}R^{b})ₜ₋₁—(CR^{c}═CR^{d})—(CR^{a}R^{b})ᵥ₋₁—, —(CR^{a}R^{b})ₜ₋₁—(C≡C)—(CR^{a}R^{b})ᵥ₋₁—, —O—, —S—, —S(O)—, —SO₂—, —C(O)—, C(O)O—, —OC(O)—, —NR^{a}—, —C(O)NR^{a}—, —NR^{a}C(O)—, —NRC(O)O—, —NR^{a}C(O)NR^{b}—, —SO₂NR^{a}—, —NR^{a}SO₂—, —NR^{a}S(O)₂NR^{b}—, —NR^{a}S(O)NR^{b}—, —C(O)NR^{a}SO₂—, —C(O)NR^{a}SO—, or —C(═NR^{a})NR^{b}—, wherein t and v, at each occurrence, are independently a number of 1 to 7, and one or two CR^{a}R^{b} moieties in —(CR^{a}R^{b})ₜ—, —(CR^{a}R^{b})ₜ₋₁—(CR^{c}═CR^{d})—(CR^{a}R^{b})ᵥ₋₁—, —(CR^{a}R^{b})ₜ₋₁—(C≡C)—(CR^{a}R^{b})ᵥ₋₁— are un-replaced or replaced with one or more moieties selected from O, S, SO, SO₂, C(O) and NR^{a};
   CyC is cycloalkyl, heterocyclyl, aryl, or heteroaryl, each of which is optionally substituted with one or two substituents R^{5a};
   R^{5a}, at each occurrence, is independently selected from hydrogen, halogen, cyano, oxo, —NO₂, —OR^{5b}, —SR^{5b}, —NR^{5b}R^{5c}, —COR^{5b}, —SO₂R^{5b}, —C(═O)OR^{5b}, —C(═O)NR^{5b}R^{5c}, —C(═NR^{5b})NR^{5c}R^{5d}, —N(R^{5b})C(═O)R^{5c}, —N(R^{5b})C(═O)OR^{5c}, —N(R^{5b})C(O)NR^{5c}R^{5d}, —N(R^{5b})S(O)NR^{5c}R^{5d}, —N(R^{5b})S(O)₂NR^{5c}R^{5d}, —NR^{5b}SO₂R^{5c}, —C₁₋₈alkyl, —C₂₋₈alkenyl, —C₂₋₈alkynyl, -cycloalkyl, heterocyclyl, aryl, or heteroaryl, each of said —C₁₋₈alkyl, —C₂₋₈alkenyl, —C₂₋₈alkynyl, -cycloalkyl, heterocyclyl, aryl, or heteroaryl is optionally substituted with one or two substituents R^{5c};
      wherein R^{5b}, R^{5c}, and R^{5d} are each independently hydrogen, —C₁₋₈alkyl, —C₂₋₈ alkenyl, —C₂₋₈alkynyl, cycloalkyl, heterocyclyl, aryl, or heteroaryl, each of said —C₁₋₈alkyl, —C₂₋₈alkenyl, C₂₋₈alkynyl, -cycloalkyl, heterocyclyl, aryl, or heteroaryl is optionally substituted with one or two substituents R^{5e};
      R^{5e}, at each occurrence, is independently selected from hydrogen, halogen, cyano, oxo, —NO₂, —OR^{5f}, —SR^{5f}, —NR^{5f}R^{5g}, —COR^{5f}, —SO₂R^{5f}, —C(═O)OR^{5f}, —C(═O)NR^{5f}R^{5g}, —C(═NR^{5f})NR^{5g}R^{5h}, —N(R^{5f})C(═O)R^{5g}, —N(R^{5f})C(═O)OR^{5g}, —N(R^{5f})C(O)NR^{5g}R^{5h}, —N(R^{5f})S(O)NR^{5g}R^{5h}, —N(R^{5f})S(O)₂NR^{5g}R^{5h}, —NR^{5f}SO₂R^{5g}, —C₁₋₈alkyl, —C₂₋₈alkenyl, —C₂₋₈alkynyl, -cycloalkyl, heterocyclyl, aryl, or heteroaryl;
         R^{5f}, R^{5g}, and R^{5h} are each independently hydrogen, —C₁₋₈alkyl, —C₂₋₈alkenyl, —C₂₋₈alkynyl, cycloalkyl, heterocyclyl, aryl, or heteroaryl;
or, two adjacent R⁵ on the phenyl ring together with the phenyl ring form a benzo ring, said ring is optionally substituted with halogen, oxo, cyano, —NO₂, —OR⁵ⁱ, —SR⁵ⁱ, —NR⁵ⁱR^{5j}, —COR⁵ⁱ, —SO₂R⁵ⁱ, —C(═O)OR⁵ⁱ, —C(═O)NR⁵ⁱR^{5j}, —C(═NR⁵ⁱ)NR^{5j}R^{5k}, —N(R⁵ⁱ)C(═O)R^{5j}, —N(R⁵ⁱ)C(═O)OR^{5j}, —N(R⁵ⁱ)C(O)NR^{5j}R^{5k}, —N(R⁵ⁱ)S(O)NR^{5j}R^{5k}, —N(R⁵ⁱ)S(O)₂NR⁵ⁱR^{5k}, —NR⁵ⁱSO₂R^{5k}, —C₁₋₈alkyl, —C₂₋₈alkenyl, —C₂₋₈alkynyl, -cycloalkyl, heterocyclyl, aryl, or heteroaryl;
   R⁵ⁱ, R^{5j}, and R^{5k} are independently hydrogen, —C₁₋₈alkyl, —C₂₋₈alkenyl, —C₂₋₈ alkynyl, cycloalkyl, heterocyclyl, aryl, or heteroaryl, each of said —C₁₋₈alkyl, —C₂₋₈alkenyl, —C₂₋₈alkynyl, cycloalkyl, heterocyclyl, aryl, or heteroaryl is optionally substituted with halogen, hydroxy or —C₁₋₈alkyoxy;
R^{a}, R^{b}, R^{c}, and R^{d} at each occurrence, are independently hydrogen, —C₁₋₈alkyl, —C₂₋₈ alkenyl, —C₂₋₈alkynyl, cycloalkyl, heterocyclyl, aryl, or heteroaryl, said —C₁₋₈alkyl, —C₂₋₈ alkenyl, —C₂₋₈alkynyl, cycloalkyl, heterocyclyl, aryl, or heteroaryl are each independently substituted with —CN, halogen, —NO₂, —NR^{e}R^{f}, oxo, —OR^{e}, or —SR^{e}; and
wherein R^{e} and R^{f} are each independently hydrogen, C₁₋₈alkyl, C₁₋₈alkoxy-C₁₋₈alkyl-, C₂₋₈alkenyl, C₂₋₈alkynyl, cycloalkyl, aryl, heterocyclyl, or heteroaryl.

In a second aspect, disclosed herein is a Bcl-2 inhibitor of Formulas (III-B), (III-C), (III-D) or (III-E) or a stereoisomer thereof, or a pharmaceutically acceptable salt thereof, in combination with azacitidine for use in the treatment of myeloid malignancies.

In a third aspect, disclosed herein is a method of treating myeloid malignancies in a subject, said method comprising administering to the subject a therapeutically effective amount of a Bcl-2 inhibitor of Formulas (III-B), (III-C), (III-D) or (III-E) or a stereoisomer thereof, or a pharmaceutically acceptable salt thereof, in combination with a therapeutically effective amount of azacitidine.

In a fourth aspect, disclosed herein is a use of a pharmaceutical composition in the manufacture of a medicament for use in the treatment of myeloid malignancies, said pharmaceutical combination comprising a Bcl-2 inhibitor of Formulas (III-B), (III-C), (III-D) or (III-E) or a stereoisomer thereof, or a pharmaceutically acceptable salt thereof, and azacitidine.

In an embodiment of each of the above aspects, the Bcl-2 inhibitor is 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide (Compound A) or a pharmaceutically acceptable salt thereof.

In one embodiment of each of the above aspects, the myeloid malignancies is acute myeloid leukemia (AML), myelodysplastic syndrome (MDS), or MDS/myeloproliferative neoplasm (MPN). In some preferred embodiment, the acute myeloid leukemia (AML) is either treatment-naïve (TN) unfit for intensive induction chemotherapy or relapsed/refractory (R/R).

In one embodiment of each of the above aspects, the Bcl-2 inhibitor is orally administrated at a dose of 40 to 160 mg once daily. In some embodiment, the Bcl-2 inhibitor is orally administrated at a dose of 40 mg, 80 mg or 160 mg daily.

In one embodiment of each of the above aspects, the Bcl-2 inhibitor is orally administrated in two Cycles, comprising a ramp-up schedule (Cycle 1) and a subsequent cycles (Cycle 2). In one embodiment of each of the above aspects, the Bcl-2 inhibitor is orally administrated in two Cycles, comprising a 4-day ramp-up schedule (Cycle 1) and a subsequent cycles (Cycle 2).

In some embodiments, each of the 4-day ramp-up schedule (Cycle 1) and the subsequent cycles (Cycle 2) is a 10-day cycle, 21-day cycle, or 28-day cycle.

In some embodiments, each of the 4-day ramp-up schedule (Cycle 1) is a 10-day cycle, 21-day cycle, or 28-day cycle, and the subsequent cycles (Cycle 2) is a 10-day cycle, 21-day cycle, or 28-day cycle.

In some preferred embodiments, each of the 4-day ramp-up schedule (Cycle 1) is a 10-day cycle, and the subsequent cycles (Cycle 2) is a 10-day cycle. In some preferred embodiments, each of the 4-day ramp-up schedule (Cycle 1) is a 28-day cycle, and the subsequent cycles (Cycle 2) is a 28-day cycle. In some preferred embodiments, each of the 4-day ramp-up schedule (Cycle 1) is a 21-day cycle, and the subsequent cycles (Cycle 2) is a 21-day cycle.

In some embodiments, the 4-day ramp-up schedule (Cycle 1) of the Bcl-2 inhibitor administration comprises the first dose at Day 1, the second dose on Day 2, the third dose on Day 3, and a recommended dose on Day 4 and beyond, wherein the recommended dose on Day 4 and beyond is higher than the second dose on Day 3, the third dose on Day 3 is higher than the second dose on Day 2, and the second dose on Day 2 is higher than the second dose on Day 1. In some more preferred embodiments, the recommended dose is 40 mg, 80 mg or 160 mg daily, and the Bcl-2 inhibitor is orally administrated at a 4-day ramp-up schedule comprising the first dose on Day 1 at 12.5% of the recommended dose, the second dose on Day 2 at 25% of recommended dose, the third dose on Day 3 at 50% of recommended dose, and the daily dose on Day 4 and beyond at 1⁰⁰% of the recommended dose. In some more preferred embodiments, the first dose on Day 1 is about 5, 10 or 20 mg/day, the second dose on Day 2 is about 10, 20 or 40 mg/day, the third dose on Day 3 is about 20, 40 or 80 mg/day, and the daily dose on Day 4 and beyond is about 40, 80 or 160 mg/day.

In some more preferred embodiments, the first dose on Day 1 is about 5 mg/day, the second dose on Day 2 is about 10 mg/day, the third dose on Day 3 is about 20 mg/day, and the daily dose on Day 4 and beyond is about 40 mg/day. In some more preferred embodiments, the first dose on Day 1 is about 10 mg/day, the second dose on Day 2 is about 20 mg/day, the third dose on Day 3 is about 40 mg/day, and the daily dose on Day 4 and beyond is about 80 mg/day. In some more preferred embodiments, the first dose on Day 1 is about 20 mg/day, the second dose on Day 2 is about 40 mg/day, the third dose on Day 3 is about 80 mg/day, and the daily dose on Day 4 and beyond is about 160 mg/day.

In some embodiments, the subsequent cycle (Cycle 2) of the Bcl-2 inhibitor administration is at a dose of a recommended dose from Day 1 with no ramp-up. In some more preferred embodiments, the recommended dose is 40 mg, 80 mg or 160 mg daily.

In some embodiments, azacitidine in Cycle 1 is administrated 1 day prior to the administration of the Bcl-2 inhibitor (designated as Day 0).

In some embodiments, azacitidine in Cycle 2 is administrated concurrently with the Bcl-2 inhibitor (designated as Day 1).

In one embodiment of each of the above aspects, azacitidine is administrated at a dose of 75 mg/m² intravenously or subcutaneously once daily (QD).

In one embodiment of each of the above aspects, azacitidine is administrated at for 7 days in Cycle 1 and Cycle 2.

In one embodiment of each of the above aspects, the Bcl-2 is orally administrated once daily (QD).

### BRIEF DESCRIPTION OF THE DRAWINGS

FIG. 1: Dosing Schema in Part 1 AML.

FIG. 2: Summary of Complete Responses in Patients with AML.

FIG. 3: Best overall response in Patients with AML.

FIG. 4: Best change from baseline in bone marrow blasts in Patients with AML.

### DEFINITIONS

Unless specifically defined elsewhere in this document, all other technical and scientific terms used herein have the meaning commonly understood by one of ordinary skill in the art.

As used herein, including the appended claims, the singular forms of words such as “a,” “an,” and “the,” include their corresponding plural references unless the context clearly dictates otherwise.

The term “or” is used to mean, and is used interchangeably with, the term “and/or” unless the context clearly dictates otherwise.

The term “anti-cancer agent” as used herein refers to any agent that can be used to treat a cell proliferative disorder such as cancer, including but not limited to, cytotoxic agents, chemotherapeutic agents, radiotherapy and radiotherapeutic agents, targeted anti-cancer agents, and immunotherapeutic agents.

The terms “administration,” “administering,” “treating,” and “treatment” herein, when applied to an animal, human, experimental subject, cell, tissue, organ, or biological fluid, means contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human. Treating any disease or disorder refer in one aspect, to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter), or both. In yet another aspect, “treat,” “treating,” or “treatment” refers to preventing or delaying the onset or development or progression of the disease or disorder.

The term “subject” in the context of the present disclosure is a mammal, e.g., a primate, preferably a higher primate, e.g., a human (e.g., a patient having, or at risk of having, a disorder described herein). In some embodiments, the subject is a human or a patient.

The terms “cancer” or “tumor” herein has the broadest meaning as understood in the art and refers to the physiological condition in mammals that is typically characterized by unregulated cell growth. In the context of the present disclosure, the cancer is not limited to a certain type or location.

The term “therapeutically effective amount” as herein used, refers to the amount of a Bcl-2 inhibitor that, when administered to a subject for treating a disease, or at least one of the clinical symptoms of a disease or disorder, is sufficient to effect such treatment for the disease, disorder, or symptom. The “therapeutically effective amount” can vary with the agent, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination objects for the effective treatment of a disease, a disorder or a condition.

The term “ramp-up scheme” or “ramp-up schedule” as herein used, refer to a dosing scheme or schedule wherein the active ingredient of interest is administrated at a dose increased at a regular basis such as daily or weekly for a designated period such as server days or several weeks, and then is administrated to the recommended dose (daily or weekly).

### DETAILED DESCRIPTION OF THE DISCLOSURE

The present disclosure provides a method of treating myeloid malignancies in a subject, comprising administering to the subject a therapeutically effective amount of a Bcl-2 inhibitor, or a stereoisomer thereof, or a pharmaceutically acceptable salt thereof, in combination with a therapeutically effective amount of azacitidine.

### Bcl-2 Inhibitor

The Bcl-2 inhibitor in the present disclosure is a compound represented by the following Formulas (III-B) (III-C) (III-D) or (III-E)

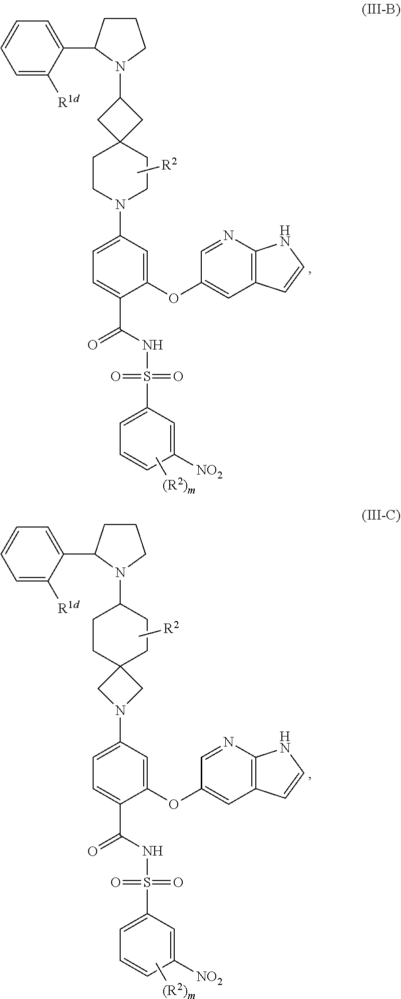

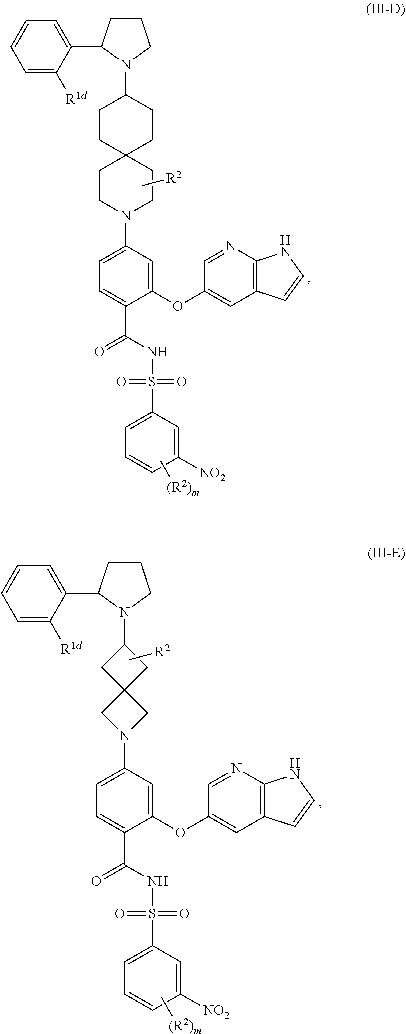

or a pharmaceutically acceptable salt thereof, or a stereoisomer thereof,
wherein,
R², at each occurrence, is independently selected from the group consisting of hydrogen, halogen, or —C₁₋₈alkyl optionally substituted with halogen;
R^{1d}, at each occurrence, is independently halogen, —C₁₋₈alkyl, —C₂₋₈alkenyl, —C₂₋₈alkynyl, cycloalkyl, heterocyclyl, aryl, heteroaryl, oxo, —CN, —NO₂, —OR^{Ba}, —SO₂R^{Ba}, —COR^{Ba}, —CO₂R^{Ba}, —CONR^{Ba}R^{Bb}, —C(═NR^{Ba})NR^{Bb}R^{Bc}, —NR^{Ba}R^{Bb}, —NR^{Ba}COR^{Bb}, —NR^{Ba}CONR^{Bb}R^{Bc}, —NR^{Ba}CO₂R^{Bb}, —NR^{Ba}SONR^{Bb}R^{Bc}, —NR^{Ba}SO₂NR^{Bb}R^{Bc}, or —NR^{a}SO₂R^{Bb}; wherein said —C₁₋₈alkyl, —C₂₋₈alkenyl, —C₂₋₈alkynyl, cycloalkyl, heterocyclyl, aryl or heteroaryl are each independently optionally substituted with 1 to 4 substituents R^{Bd};
R^{Ba}, R^{Bb}, and R^{Bc}, are each independently hydrogen, —C₁₋₈alkyl, —C₂₋₈alkenyl, —C₂₋₈alkynyl, cycloalkyl, heterocyclyl, aryl, or heteroaryl, each of said —C₁₋₈alkyl, —C₂₋₈alkenyl, —C₂₋₈ alkynyl, cycloalkyl, heterocyclyl, aryl, or heteroaryl is optionally substituted with halogen, hydroxy, —NH₂ or —N(C₁₋₆alkyl)₂, —C₁₋₈alkyoxy, cycloalkyl, heterocyclyl, aryl, or heteroaryl;
R^{Bd}, at each occurrence, is independently hydrogen, halogen, oxo, —CN, —NO₂, —C₁₋₈alkyl, —C₂₋₈alkenyl, —C₂₋₈alkynyl, cycloalkyl, heterocyclyl, aryl, or heteroaryl, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, —C₁₋₈alkyoxy, cycloalkyl, heterocyclyl, aryl, or heteroaryl;
m is an integer of 1-4;
R⁵ is -L⁵-CyC,
   Wherein L⁵ is a direct bond, —(CR^{a}R^{b})ₜ—, —(CR^{a}R^{b})ₜ₋₁—(CR^{c}═CR^{d})—(CR^{a}R^{b})ᵥ₋₁—, —(CR^{a}R^{b})ₜ₋₁—(C≡C)—(CR^{a}R^{b})ᵥ₋₁—, —O—, —S—, —S(O)—, —SO₂—, —C(O)—, C(O)O—, —OC(O)—, —NR^{a}—, —C(O)NR^{a}—, —NR^{a}C(O)—, —NRC(O)O—, —NR^{a}C(O)NR^{b}—, —SO₂NR^{a}—, —NR^{a}SO₂—, —NR^{a}S(O)₂NR^{b}—, —NR^{a}S(O)NR^{b}—, —C(O)NR^{a}SO₂—, —C(O)NR^{a}SO—, or —C(═NR^{a})NR^{b}—, wherein t and v, at each occurrence, are independently a number of 1 to 7, and one or two CR^{a}R^{b} moieties in —(CR^{a}R^{b})ₜ—, —(CR^{a}R^{b})ₜ₋₁—(CR^{c}═CR^{d})—(CR^{a}R^{b})ᵥ₋₁—, —(CR^{a}R^{b})ₜ₋₁—(C≡C)—(CR^{a}R^{b})ᵥ₋₁— are un-replaced or replaced with one or more moieties selected from O, S, SO, SO₂, C(O) and NR^{a};
   CyC is cycloalkyl, heterocyclyl, aryl, or heteroaryl, each of which is optionally substituted with one or two substituents R^{5a};
   R^{5a}, at each occurrence, is independently selected from hydrogen, halogen, cyano, oxo, —NO₂, —OR^{5b}, —SR^{5b}, —NR^{5b}R^{5c}, —COR^{5b}, —SO₂R^{5b}, —C(═O)OR^{5b}, —C(═O)NR^{5b}R^{5c}, —C(═NR^{5b})NR^{5c}R^{5d}, —N(R^{5b})C(═O)R^{5c}, —N(R^{5b})C(═O)OR^{5c}, —N(R^{5b})C(O)NR^{5b}R^{5d}, —N(R^{5b})S(O)NR^{5c}R^{5d}, —N(R^{5b})S(O)₂NR^{5c}R^{5d}, NR^{5b}SO₂R^{5c}, —C₁₋₈alkyl, —C₂₋₈alkenyl, —C₂₋₈alkynyl, -cycloalkyl, heterocyclyl, aryl, or heteroaryl, each of said —C₁₋₈alkyl, —C₂₋₈alkenyl, —C₂₋₈alkynyl, -cycloalkyl, heterocyclyl, aryl, or heteroaryl is optionally substituted with one or two substituents R^{5c};
      wherein R^{5b}, R^{5c}, and R^{5d} are each independently hydrogen, —C₁₋₈alkyl, —C₂₋₈ alkenyl, —C₂₋₈alkynyl, cycloalkyl, heterocyclyl, aryl, or heteroaryl, each of said —C₁₋₈alkyl, —C₂₋₈alkenyl, C₂₋₈alkynyl, -cycloalkyl, heterocyclyl, aryl, or heteroaryl is optionally substituted with one or two substituents R^{5e};
      R^{5e}, at each occurrence, is independently selected from hydrogen, halogen, cyano, oxo, —NO₂, —OR^{5f}, —SR^{5f}, —NR^{5f}R^{5g}, —COR^{5f}, —SO₂R^{5f}, —C(═O)OR^{5f}, —C(═O)NR^{5f}R^{5g}, —C(═NR^{5f})NR^{5g}R^{5h}, —N(R^{5f})C(═O)R^{5g}, —N(R^{5f})C(═O)OR^{5g}, —N(R^{5f})C(O)NR^{5g}R^{5h}, —N(R^{5f})S(O)NR^{5g}R^{5h}, —N(R^{5f})S(O)₂NR^{5g}R^{5h}, —NR^{5f}SO₂R^{5g}, —C₁₋₈alkyl, —C₂₋₈alkenyl, —C₂₋₈alkynyl, -cycloalkyl, heterocyclyl, aryl, or heteroaryl;
         R^{5f}, R^{5g}, and R^{5h} are each independently hydrogen, —C₁₋₈alkyl, —C₂₋₈alkenyl, —C₂₋₈alkynyl, cycloalkyl, heterocyclyl, aryl, or heteroaryl;
   or, two adjacent R⁵ on the phenyl ring together with the phenyl ring form a benzo ring, said ring is optionally substituted with halogen, oxo, cyano, —NO₂, —OR⁵ⁱ, —SR⁵ⁱ, —NR⁵ⁱR^{5j}, —COR⁵ⁱ, —SO₂R⁵ⁱ, —C(═O)OR⁵ⁱ, —C(═O)NR⁵ⁱR^{5j}, —C(═NR⁵ⁱ)NR^{5j}R^{5k}, —N(R⁵ⁱ)C(═O)R^{5j}, —N(R⁵ⁱ)C(═O)OR^{5j}, —N(R⁵ⁱ)C(O)NR^{5j}R^{5k}, —N(R⁵ⁱ)S(O)NR^{5j}R^{5k}, —N(R⁵ⁱ)S(O)₂NR⁵ⁱR^{5k}, —NR⁵ⁱSO₂R^{5k}, —C₁₋₈alkyl, —C₂₋₈alkenyl, —C₂₋₈alkynyl, -cycloalkyl, heterocyclyl, aryl, or heteroaryl;
      R⁵ⁱ, R^{5j}, and R^{5k} are independently hydrogen, —C₁₋₈alkyl, —C₂₋₈alkenyl, —C₂₋₈ alkynyl, cycloalkyl, heterocyclyl, aryl, or heteroaryl, each of said —C₁₋₈alkyl, —C₂₋₈alkenyl, —C₂₋₈alkynyl, cycloalkyl, heterocyclyl, aryl, or heteroaryl is optionally substituted with halogen, hydroxy or —C₁₋₈alkyoxy;
   R^{a}, R^{b}, R^{c}, and R^{d} at each occurrence, are independently hydrogen, —C₁₋₈alkyl, —C₂₋₈ alkenyl, —C₂₋₈alkynyl, cycloalkyl, heterocyclyl, aryl, or heteroaryl, said —C₁₋₈alkyl, —C₂₋₈ alkenyl, —C₂₋₈alkynyl, cycloalkyl, heterocyclyl, aryl, or heteroaryl are each independently substituted with —CN, halogen, —NO₂, —NR^{e}R^{f}, oxo, —OR^{e}, or —SR^{e}; and wherein R^{e} and R^{f} are each independently hydrogen, C₁₋₈alkyl, C₁₋₈alkoxy-C₁₋₈alkyl-, C₂₋₈ alkenyl, C₂₋₈alkynyl, cycloalkyl, aryl, heterocyclyl, or heteroaryl.

In some embodiments, R² is hydrogen.

In some embodiments, R^{1d}, when substituted on the phenyl group at position 2 of ring B (including the aziridin-1-yl, azetidin-1-yl, pyrrolidin-1-yl, pyrrolidin-2-yl, piperidin-1-yl, azepan-1-yl, or azocan-1-yl, preferably the pyrrolidin-1-yl group), is independently halogen, —C₁₋₈alkyl, —C₂₋₈alkenyl, —C₂₋₈alkynyl, cycloalkyl, heterocyclyl, aryl, heteroaryl, —CN, —OR^{Ba}, —SO₂R^{Ba}, —CONR^{Ba}R^{Bb}, —NO₂, —NR^{Ba}R^{Bb}, —NR^{Ba}COR^{Bb}, or —NR^{Ba}SO₂R^{Bb}; wherein said —C₁₋₈alkyl, —C₂₋₈alkenyl, —C₂₋₈alkynyl, cycloalkyl, heterocyclyl, aryl or heteroaryl are each independently optionally substituted with 1 to 4 substituents R^{Bd} as defined as with Formulas (III-B), (III-C), (III-D) or (III-E), preferably 1 or 2 substituents R^{Bd} as defined as with Formulas (III-B), (III-C), (III-D) or (III-E). In another aspect, one R^{1d} is at position 2 of the phenyl ring at position 2 of ring B.

In some embodiments, R^{1d} is methyl, ethyl, isopropyl, propyl or methoxymethyl, or two methyl at the position of the phenyl ring; or propenyl; or cyclopropyl, cyclobutyl, cyclopentyl, or cyclohexyl; or ethoxy or isopropoxy; or amino or dimethylamino.

In some embodiments, the 2-(2-substituted phenyl)pyrrolidin-1-yl moiety in Formulas (III-B), (III-C), (III-D) or (III-E), is selected from the group consisting of:

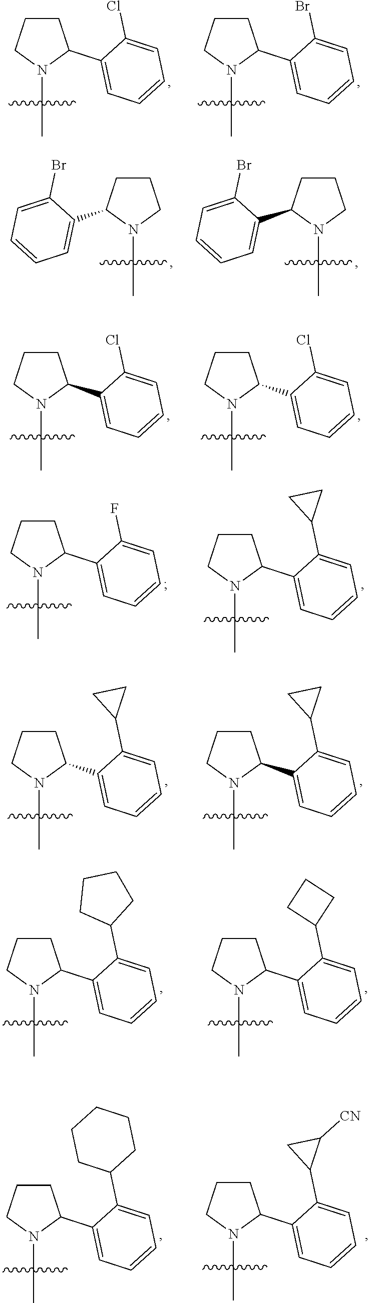

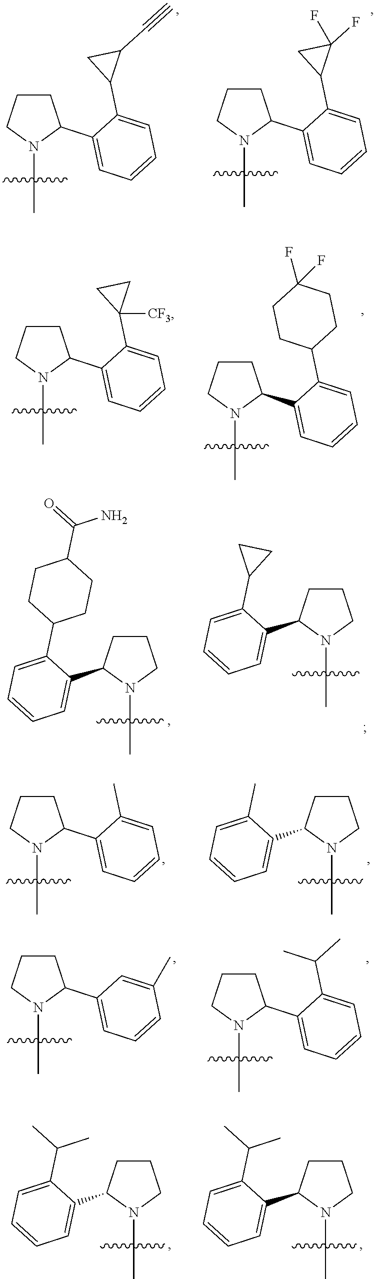

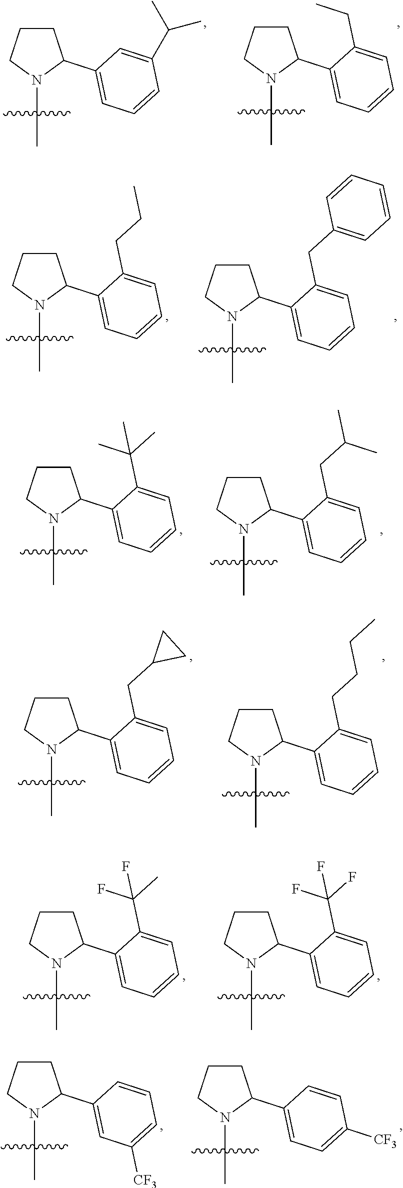

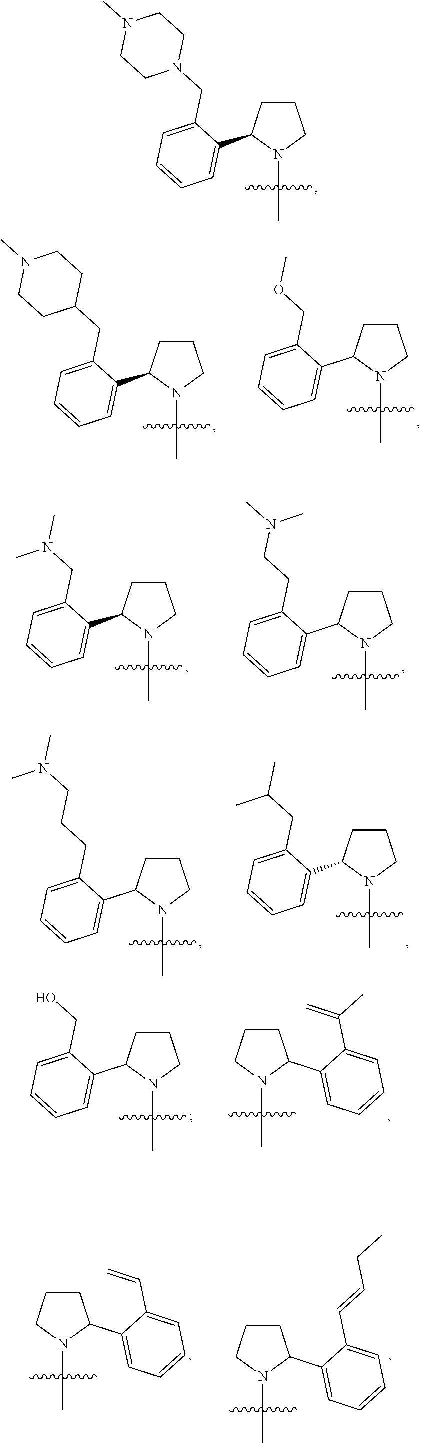

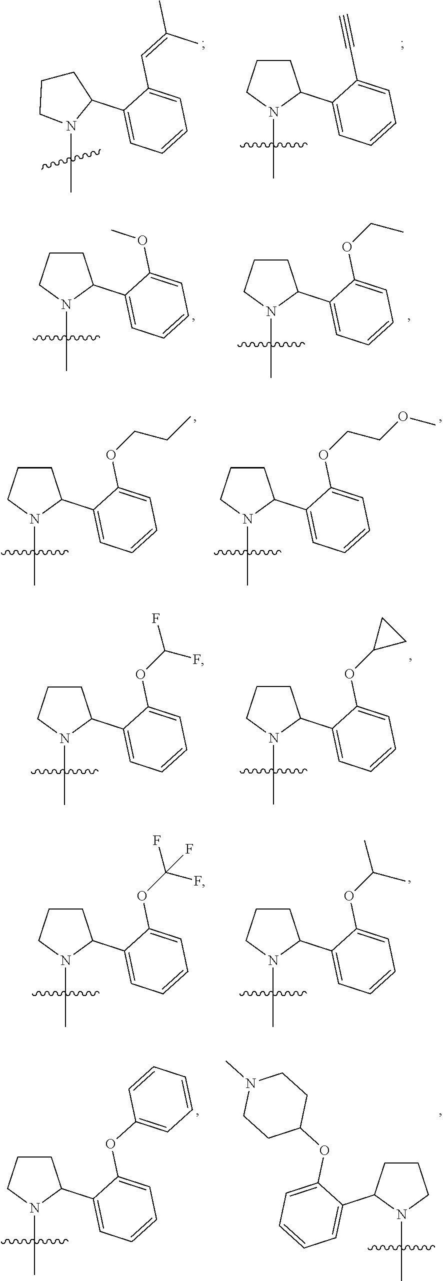

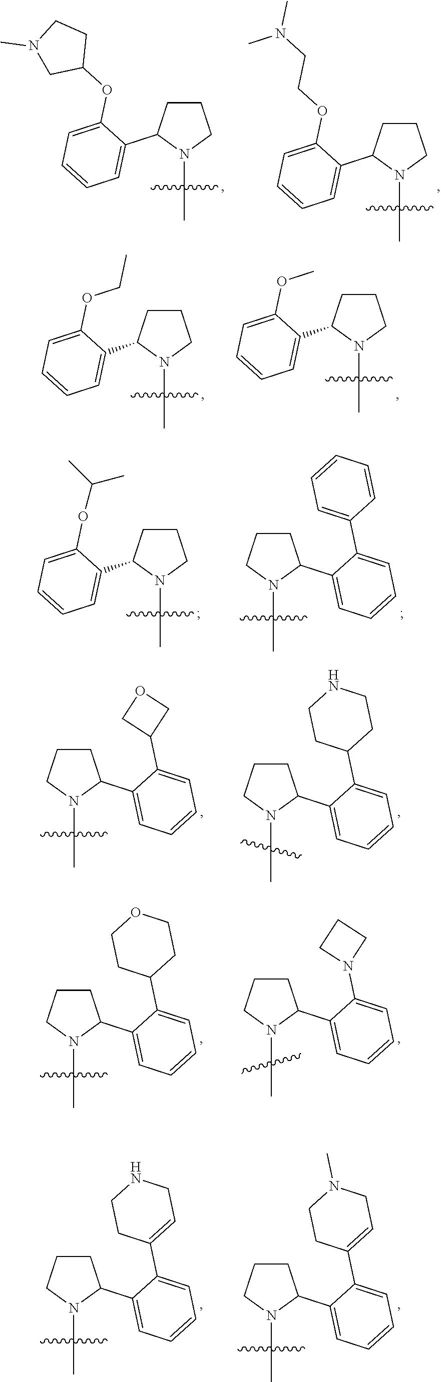

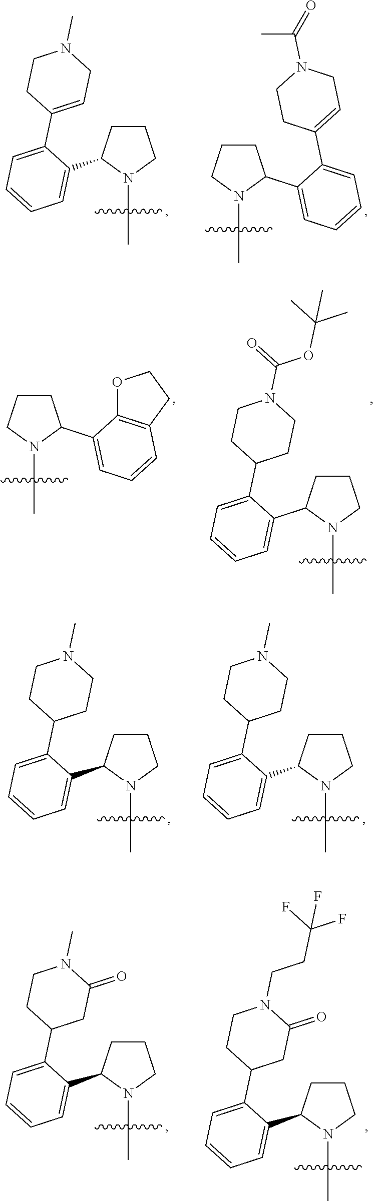

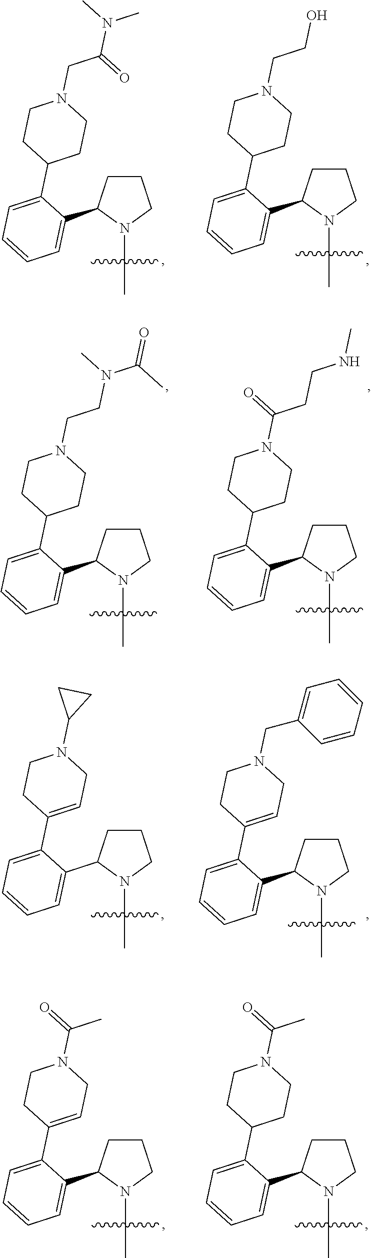

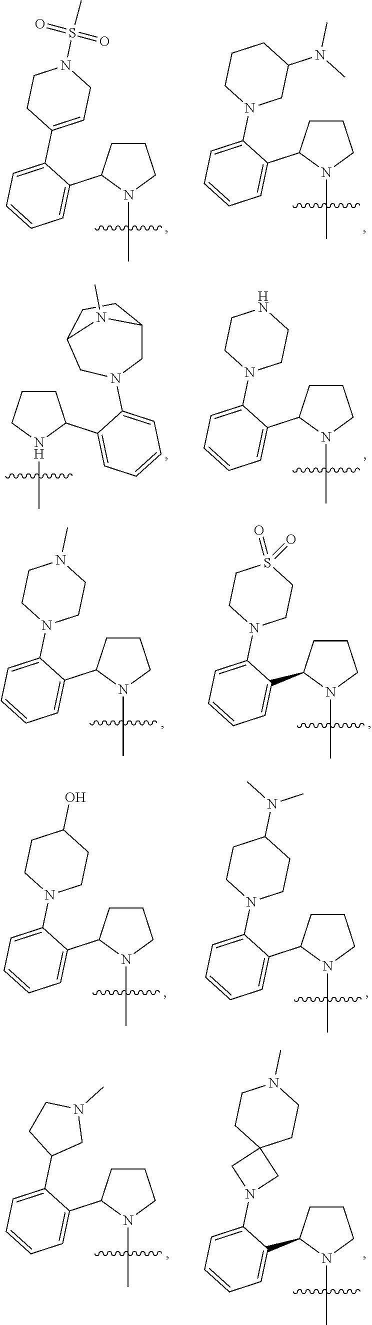

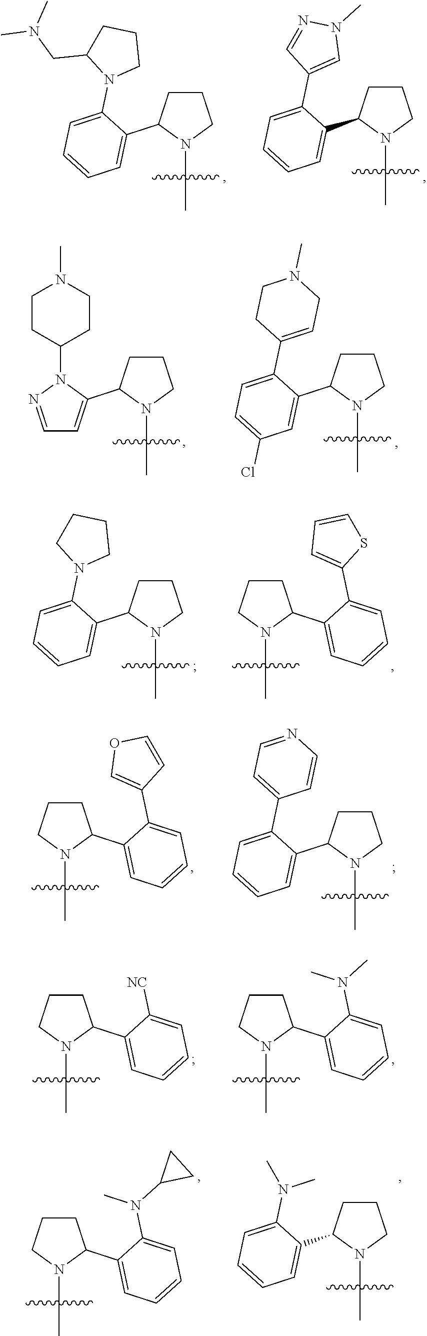

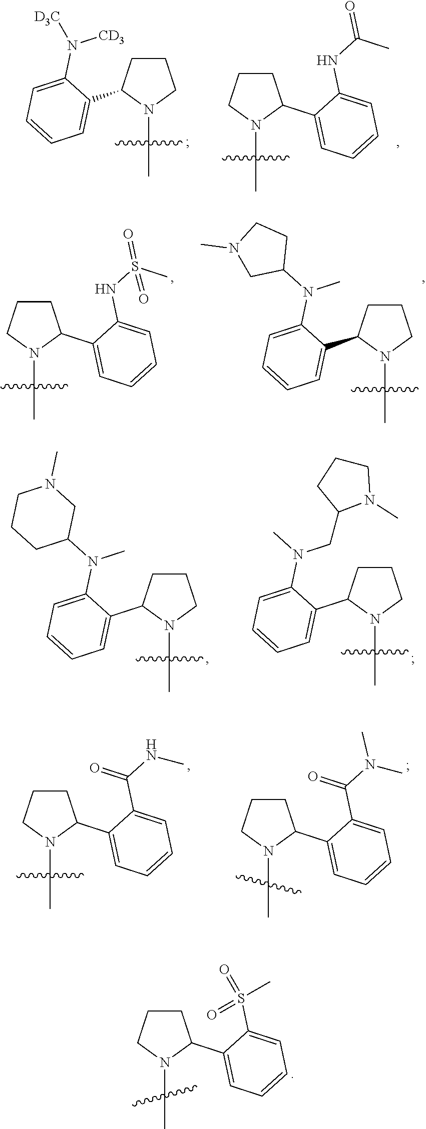

In some embodiments, m is 1; and L⁵ is a direct bond, —(CR^{a}R^{b})ₜ— or —NR^{a}—, wherein t is a number of 1 to 7, and one or two CR^{a}R^{b} moieties in —(CR^{a}R^{b})ₜ— are un-replaced or replaced with one or more moieties selected from O and NR^{a}, wherein R^{a} and R^{b} are defined with Formulas (III-B), (III-C), (III-D) or (III-E).

In some embodiments, L⁵ is a direct bond, —(CR^{a}R^{b})₁₋₄—, —O—(CR^{a}R^{b})₁₋₃, —NH—(CR^{a}R^{b})₁₋₃, or —NH—, wherein R^{a} and R^{b} are defined as with Formulas (III-B), (III-C), (III-D) or (III-E), so that the -L⁵-CyC moiety is CyC, —(CR^{a}R^{b})₁₋₄-CyC, —O—(CR^{a}R^{b})₁₋₃-CyC, —NH—(CR^{a}R^{b})₁₋₃-CyC, or —NH-CyC, respectively. More preferably, L⁵ is a direct bond, —(CH₂)₁₋₄—, —O—(CH₂)₁₋₃—, —NH—(CR^{a}R^{b})—(CH₂)₂—, or —NH—, wherein R^{b} is hydrogen and R^{b} is C₁₋₈alkyl optionally substituted with phenyl-S— so that the -L⁵-CyC moiety is CyC, —(CH₂)₁₋₄-CyC, —O—(CH₂)₁₋₃-CyC, —NH—(CR^{a}R^{b})—(CH₂)₂-CyC, or —NH-CyC, respectively. More preferably, L³ is a direct bond, —CH₂—, —O—CH₂—, —NH—CH₂—, or —NH— so that the -L⁵-CyC moiety is CyC, —CH₂—CyC, —O—CH₂-CyC, —NH—CH₂—CyC, or —NH-CyC, respectively.

In some embodiments, CyC is cycloalkyl, or heterocyclyl, each of which is optionally substituted with one or two substituents R^{5a};
R^{5a} is independently selected from hydrogen, halogen, cyano, oxo, —OR^{5b}, —NR^{5b}R^{5c}, —COR^{5b}, —SO₂R^{5b}, —C₁₋₈alkyl, —C₂₋₈alkynyl, -cycloalkyl, or heterocyclyl, each of said —C₁₋₈alkyl, and heterocyclyl is optionally substituted with one or two substituents R^{5e} which is selected from hydrogen, halogen, cyano, —OR^{5f}, —C₁₋₈alkyl, -cycloalkyl, or heterocyclyl;
   wherein R^{5b}, and R^{5c} are each independently hydrogen, —C₁₋₈alkyl or heterocyclyl, said —C₁₋₈alkyl is optionally substituted with one or two substituents R^{5e} which is hydrogen, —NR^{5f}R^{5g}, or -cycloalkyl;
      R^{5f} and R^{5g} are each independently hydrogen or —C₁₋₈alkyl;
   or, two adjacent R⁵ on the phenyl ring together with the phenyl ring form a benzo ring, said ring is optionally substituted with heteroaryl.

In some embodiments, CyC is cycloalkyl selected from monocyclic C₃₋₈cycloalkyl or bridged cycloalkyl

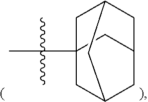 [SMILES: *C12CC3CC(CC(C3)C1)C2]

each of which is optionally substituted with one or two substituents R^{5a}. preferably, CyC is cyclopentyl or cyclohexyl, each of which is optionally substituted with one or two substituents R^{5a}.

In some embodiments, CyC is heterocyclyl selected from:
a) monocyclic 4 to 9-membered heterocyclyl groups containing one nitrogen or oxygen or sulfur heteroatom as a ring member;
b) monocyclic 4 to 9-membered heterocyclyl groups containing two heteroatoms selected from oxygen, sulfur and nitrogen as ring members; and
c) 5 to 20-membered spiro heterocyclyl comprising one or two heteroatoms selected from nitrogen, sulfur and oxygen as ring members,
each of which is optionally substituted with one or two R^{5a};

In some embodiments, CyC is monocyclic 4 to 6-membered heterocyclyl groups containing one nitrogen or oxygen or sulfur heteroatom as the ring member. More preferably, Cyc is selected from oxetanyl, tetrahydrofuranyl, tetrahydropyranyl, azetidinyl, pyrrolidinyl, and piperdinyl. Even more preferably, CyC is selected from □oxetan-2-yl, Oxetan-3-yl, tetrahydrofuran-4-yl, tetrahydrofuran-2-yl, tetrahydrofuran-3-yl, tetrahydropyran-2-yl, tetrahydropyran-3-yl, tetrahydropyran-4-yl, azetidin-3-yl, azetidin-2-yl, pyrrolidin-2-yl, pyrrolidin-3-yl, piperdin-4-yl, piperdin-2-yl, and piperdin-3-yl.

In some embodiments, CyC is a monocyclic 6-membered heterocyclyl group containing two heteroatoms selected from oxygen and nitrogen as ring members. More preferably, CyC is dioxanyl, morpholino, morpholinyl, or piperzinyl. Even more preferably 1,3-dioxan-2-yl, 1,3-dioxan-4-yl, 1,4-dioxan-2-yl, morpholin-1-yl, morpholin-2-yl, or morpholin-3-yl.

In some embodiments, R^{5a} is independently selected from hydrogen, halogen, cyano, oxo, —OR^{5b}, —NR^{5b}R^{5c}, —COR^{5b}, —SO₂R^{5b}, —C₁₋₈alkyl, —C₂₋₈alkynyl, monocyclic C₃₋₈cycloalkyl, or monocyclic 4 to 9-membered heterocyclyl group containing one or two heteroatoms selected from nitrogen or oxygen or sulfur heteroatom as ring members, each of said —C₁₋₈alkyl and monocyclic 4 to 9-membered heterocyclyl group is optionally substituted with one or two substituents R^{5e}; preferably, cycloalkyl as R^{5a} is C₃₋₆cycloalkyl; more preferably cyclopropyl; preferably, heterocyclyl as R^{5a} is 4 to 6-membered heterocyclyl groups containing one or two heteroatoms selected from nitrogen or oxygen or sulfur heteroatom as ring members; more preferably, heterocyclyl as R^{5a} is oxetanyl, tetrahydrofuranyl, tetrahydropyranyl, piperzinyl, or morpholinyl; even more preferably, heterocyclyl as R^{5a} is oxetan-3-yl, tetrahydrofuran-3-yl, tetrahydro-2H-pyran-4-yl, or morphin-4-yl.

In some embodiments, heterocyclyl as R^{5e} is a monocyclic 4 to 9-membered heterocyclyl group containing one or two heteroatoms selected from nitrogen or oxygen or sulfur heteroatom as ring members.

In some embodiments, heterocyclyl as R^{5e} is tetrahydro-pyran-4-yl.

In some embodiments, R^{5a} is —NR^{5b}R^{5c}, wherein R^{5b} is hydrogen, and R^{5c} is heterocyclyl.

In some embodiments, R^{5a} is —NR^{5b}R^{5c}, wherein R^{5b} is hydrogen, and R^{5c} is tetrahydro-pyran-4-yl.

In some embodiments, R^{5a} is —NR^{5b}R^{5c}, wherein R^{5b} and R^{5c} are each independently hydrogen or —C₁₋₆alkyl substituted with cycloalkyl, preferably —C₁₋₆alkyl substituted with monocyclic C₃₋₈cycloalkyl.

In some embodiments, R^{5a} is —OR^{5b} or —SO₂R^{5b}, wherein R^{5b} is hydrogen or C₁₋₈alkyl, preferably methyl.

In some embodiments, R^{5a} is —COR^{5b}, wherein R^{5b} is hydrogen or C₁₋₈alkyl optionally substituted with —NR^{5f}R^{5g}, wherein R^{5f} and R^{5g} are each independently hydrogen or C₁₋₈alkyl, preferably methyl.

In some embodiments, two adjacent R⁵ on the phenyl ring together with the phenyl ring form indazolyl which is substituted with tetrahydropyranyl.

In some embodiments, m is 1, and R⁵ is -L⁵-CyC selected from the group consisting of:

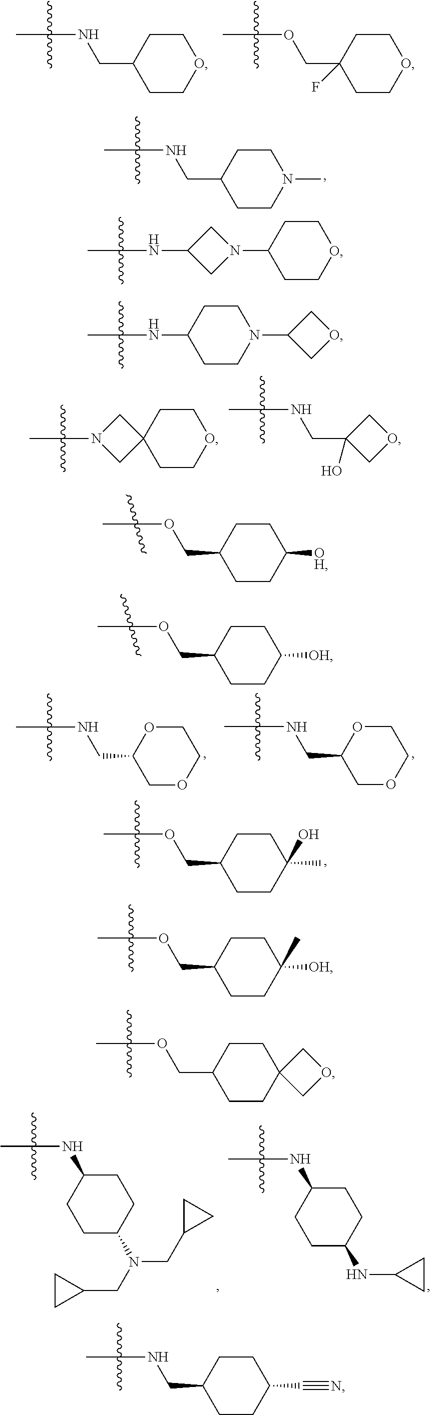

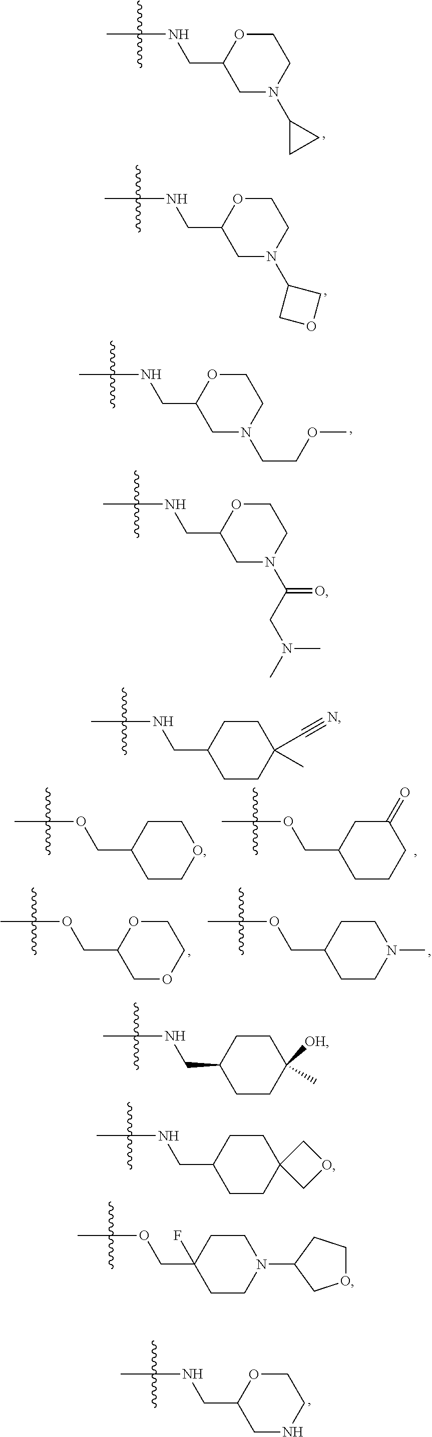

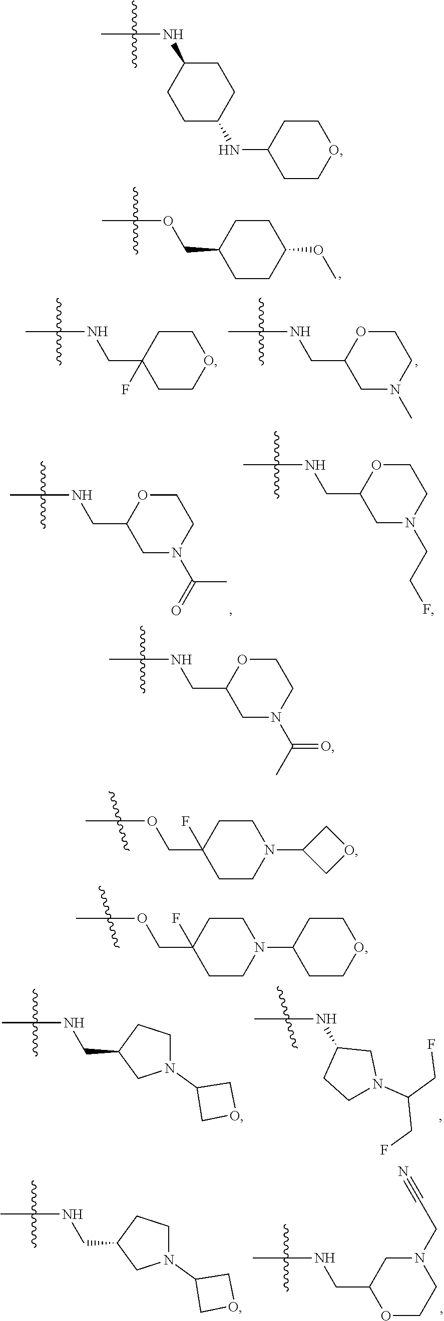

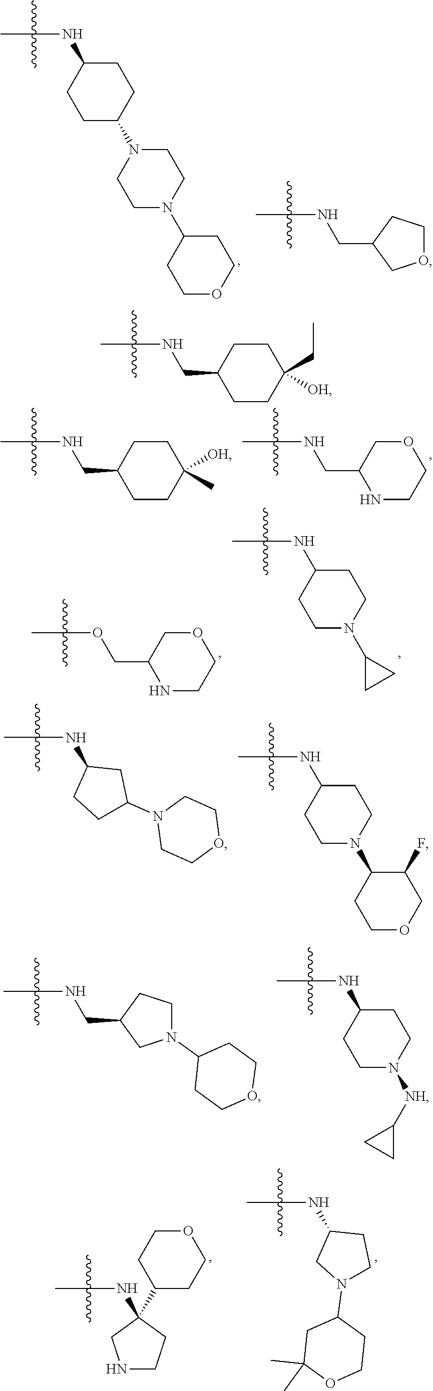

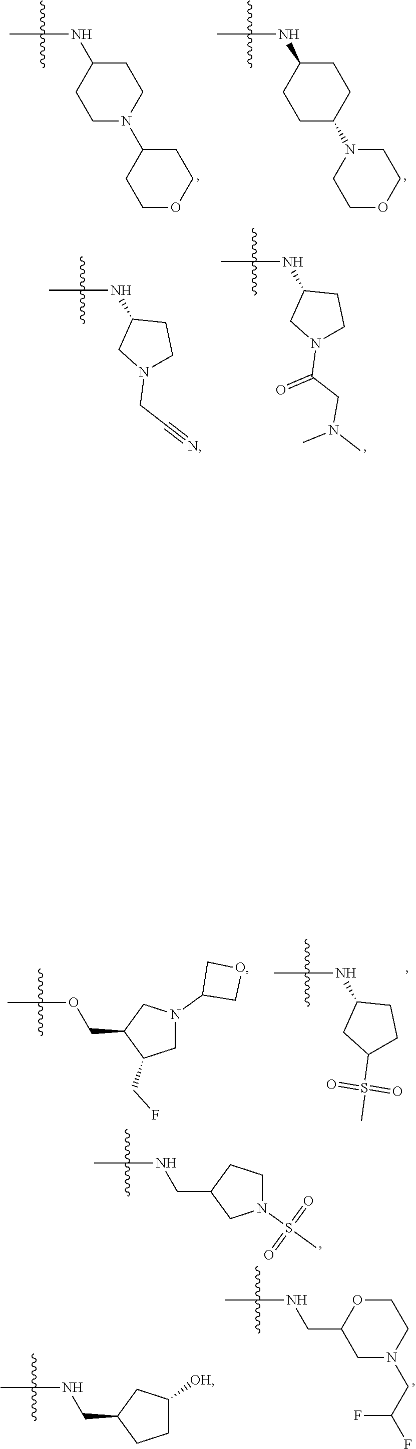

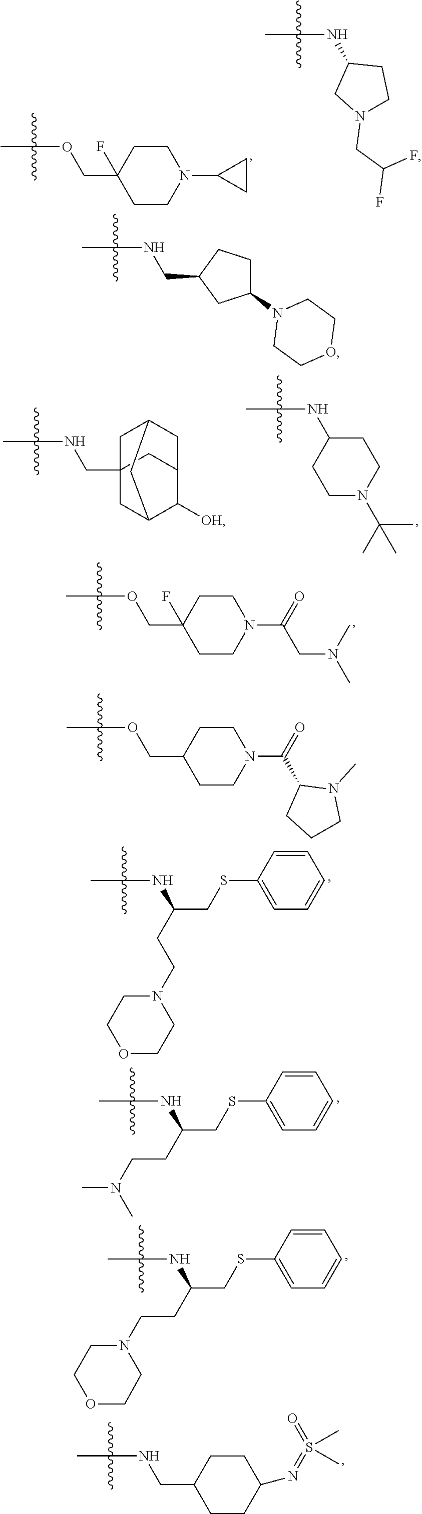

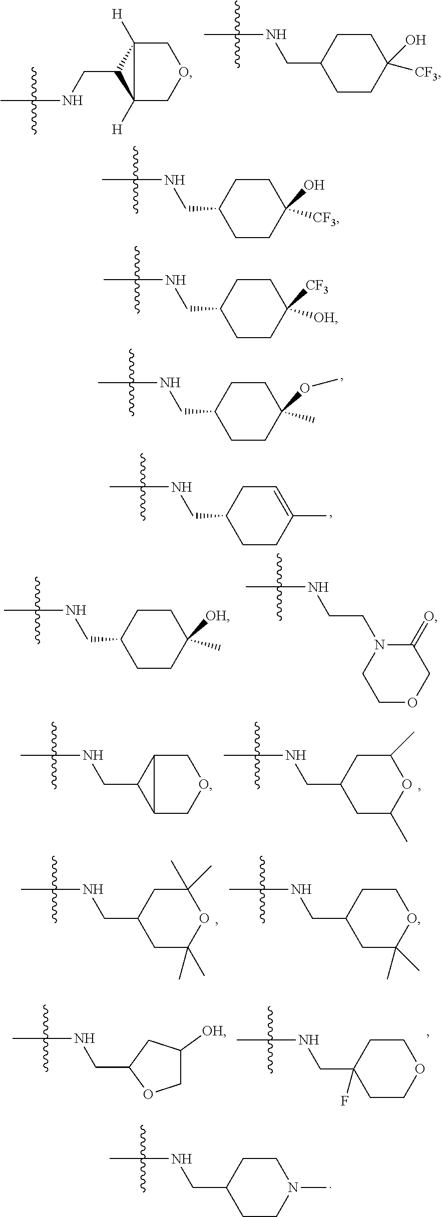

In some embodiments, m is 1 and R⁵ is

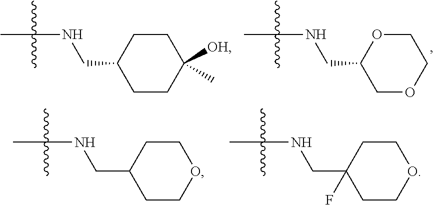 [SMILES: *NC[C@H]1CC[C@](C)(O)CC1 ; *NC[C@H]1COCCO1 ; *NCC1CCOCC1 ; *NCC1(F)CCOCC1]

In some embodiments, the Bcl-2 inhibitor in present disclosure is selected form the group consist of:
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(((4-fluorotetrahydro-2H-pyran-4-yl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-ethylphenyl)pyrrolidin-1-yl)-7-azaspiro[3.5]nonan-7-yl)-N-((4-(((4-fluorotetrahydro-2H-pyran-4-yl)methyl)amino)-3-nitrophenyl)sulfonyl)benzamide;
2-((1H-pyrrolo[2,3-b]pyridin-5-yl)oxy)-4-(2-((S)-2-(2-ethylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
(R)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(((4-fluorotetrahydro-2H-pyran-4-yl)methyl)amino)-3-nitrophenyl)sulfonyl)benzamide;
2-((1H-pyrrolo[2,3-b]pyridin-5-yl)oxy)-4-(2-((R)-2-(2-cyclopropylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
(S)-2-((1H-pyrrolo[2,3-b]pyridin-5-yl)oxy)-4-(7-(2-(2-cyclopropylphenyl)pyrrolidin-1-yl)-2-azaspiro[3.5]nonan-2-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(7-(2-(2-cyclopropylphenyl)pyrrolidin-1-yl)-2-azaspiro[3.5]nonan-2-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(9-(2-(2-cyclopropylphenyl)pyrrolidin-1-yl)-3-azaspiro[5.5]undecan-3-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(9-(2-(2-cyclopropylphenyl)pyrrolidin-1-yl)-3-azaspiro[5.5]undecan-3-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(6-(2-(2-cyclopropylphenyl)pyrrolidin-1-yl)-2-azaspiro[3.3]heptan-2-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(6-(2-(2-cyclopropylphenyl)pyrrolidin-1-yl)-2-azaspiro[3.3]heptan-2-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(3-chloro-2-(dimethylamino)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)benzamide;
(S)2-((1H-pyrrolo[2,3-b]pyridin-5-yl)oxy)-4-(2-((S)-2-(2-cyclopropylphenyl)pyrrolidin-1-yl)-8-azaspiro[4.5]decan-8-yl)-N-((3-nitro-4-(((tetrahydro-2H-pyran-4-yl)methyl)amino)phenyl)sulfonyl)benzamide;
(R) 2-((1H-pyrrolo[2,3-b]pyridin-5-yl)oxy)-4-(2-((S)-2-(2-cyclopropylphenyl)pyrrolidin-1-yl)-8-azaspiro[4.5]decan-8-yl)-N-((3-nitro-4-(((tetrahydro-2H-pyran-4-yl)methyl)amino)phenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-ethylphenyl)pyrrolidin-1-yl)-7-azaspiro[3.5]nonan-7-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-ethylphenyl)pyrrolidin-1-yl)-7-azaspiro[3.5]nonan-7-yl)benzamide;
(S)-2-((1H-pyrrolo[2,3-b]pyridin-5-yl)oxy)-4-(8-(2-(2-cyclopropylphenyl)pyrrolidin-1-yl)-2-azaspiro[4.5]decan-2-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-isopropylphenyl)pyrrolidin-yl)-7-azaspiro[3.5]nonan-7-yl)benzamide;
(S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-N-((4-(((4-fluorotetrahydro-2H-pyran-4-yl)methyl)amino)-3-nitrophenyl)sulfonyl)-4-(2-(2-(2-isopropylphenyl)pyrrolidin-1-yl)-7-azaspiro[3.5]nonan-7-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1s,4s)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((R)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1s,4s)-4-hydroxy-4-methylcyclohexyl)methyl)amino)-3-nitrophenyl)sulfonyl)-4-(2-((R)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-4-(2-(2-(2-cyclobutylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-isobutylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(3-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-N-((3-nitro-4-(((tetrahydro-2H-pyran-4-yl)methyl)amino)phenyl)sulfonyl)-4-(2-(2-(o-tolyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-bromophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(2-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(3-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(3-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(4-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-ethoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-(dimethylamino)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-((S)-2-(2-(dimethylamino)phenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(2-((S)-2-(2-(bis(methyl-d3)amino)phenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-N-((3-nitro-4-(((tetrahydro-2H-pyran-4-yl)methyl)amino)phenyl)sulfonyl)-4-(2-(2-(2-(pyrrolidin-1-yl)phenyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-(1-methyl-1,2,3,6-tetrahydropyridin-4-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-(1-methylpiperidin-4-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-methoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-(methoxymethyl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-(hydroxymethyl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(3-chloro-2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(3-chloro-2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(5-chloro-2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(3-chloro-2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S or R)-2-((1H-pyrrolo[2,3-b]pyridin-5-yl)oxy)-4-(2-(2-(3-chloro-2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2,4-dicyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2,5-dicyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(3-(2-chlorophenyl)thiophen-2-yl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-methylpyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(4-cyclopropyl-2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-phenyl-2,5-dihydro-1H-pyrrol-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4,4-dimethylpyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4,4-difluoropyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-(trifluoromethyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(2-(2-(2-cyclopropylphenyl)-4-(dimethylamino)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-(2-(dimethylamino)ethoxy)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-3,3-dimethylpyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-(((((1s,4s) or (1r,4r))-4-((dimethyl(oxo)-16-sulfaneylidene)amino)cyclohexyl)methyl)amino)-3-nitrophenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-(methyl(3-nitro-4-(((tetrahydro-2H-pyran-4-yl)methyl)amino)phenyl)(oxo)-16-sulfaneylidene)benzamide;
2-((1H-pyrrolo[2,3-b]pyridin-5-yl)oxy)-N-((4-(((-3-oxabicyclo[3.1.0]hexan-6-yl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(((4-hydroxy-4-(trifluoromethyl)cyclohexyl)methyl)amino)-3-nitrophenyl)sulfonyl)benzamide;
2-((1H-pyrrolo[2,3-b]pyridin-5-yl)oxy)-N-((4-((((1r,4r)-4-hydroxy-4-(trifluoromethyl)cyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1s,4s)-4-hydroxy-4-(trifluoromethyl)cyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)benzamide;
2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)-N-((4-((((S)-4-methylcyclohex-3-en-1-yl)methyl)amino)-3-nitrophenyl)sulfonyl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-(prop-1-en-2-yl)phenyl)pyrrolidin-1-yl)-7-azaspiro[3.5]nonan-7-yl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-propylphenyl)pyrrolidin-1-yl)-7-azaspiro[3.5]nonan-7-yl)benzamide;
2-((1H-pyrrolo[2,3-b]pyridin-5-yl)oxy)-4-(6-((S)-2-(2-cyclopropylphenyl)pyrrolidin-1-yl)-2-azaspiro[3.3]heptan-2-yl)-N-((4-((((1r,4r)-4-hydroxy-4-methylcyclohexyl)methyl)amino)-3-nitrophenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-cyclopropylphenyl)pyrrolidin-1-yl)-2-azaspiro[3.3]heptan-2-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-cyclopropylphenyl)pyrrolidin-1-yl)-2-azaspiro[3.3]heptan-2-yl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(6-((S)-2-(2-isopropylphenyl)pyrrolidin-1-yl)-2-azaspiro[3.3]heptan-2-yl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-isopropylphenyl)pyrrolidin-1-yl)-2-azaspiro[3.3]heptan-2-yl)benzamide;
2-((1H-pyrrolo[2,3-b]pyridin-5-yl)oxy)-4-(6-((S)-2-(2-ethylphenyl)pyrrolidin-1-yl)-2-azaspiro[3.3]heptan-2-yl)-N-((4-((((1r,4r)-4-hydroxy-4-methylcyclohexyl)methyl)amino)-3-nitrophenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-ethylphenyl)pyrrolidin-1-yl)-2-azaspiro[3.3]heptan-2-yl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-((2-(tetrahydro-2H-pyran-4-yl)ethyl)amino)phenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-((2-morpholinoethyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-((2-(3-oxomorpholino)ethyl)amino)phenyl)sulfonyl)benzamide;
2-((1H-pyrrolo[2,3-b]pyridin-5-yl)oxy)-N-((4-(((3-oxabicyclo[3.1.0]hexan-6-yl)methyl)amino)-3-nitrophenyl)sulfonyl)-4-(2-((S)-2-(2-cyclopropylphenyl)pyrrolidin-1-yl)-7-azaspiro[3.5]nonan-7-yl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-(((2,6-dimethyltetrahydro-2H-pyran-4-yl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-(((2,2,6,6-tetramethyltetrahydro-2H-pyran-4-yl)methyl)amino)phenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-6-azaspiro[3.4]octan-6-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(6-((S)-2-(2-cyclopropylphenyl)pyrrolidin-1-yl)-2-azaspiro[3.4]octan-2-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-((7R or 7S)-7-((S)-2-(2-cyclopropylphenyl)pyrrolidin-1-yl)-2-azaspiro[4.4]nonan-2-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-((7S or 7R)-7-((S)-2-(2-cyclopropylphenyl)pyrrolidin-1-yl)-2-azaspiro[4.4]nonan-2-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-(((2,2-dimethyltetrahydro-2H-pyran-4-yl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(3-methyl-3-((tetrahydro-2H-pyran-4-yl)methyl)ureido)-3-nitrophenyl)sulfonyl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-phenylpyrrolidin-1-yl)-7-azaspiro[3.5]nonan-7-yl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-((((cis or trans)-4-hydroxytetrahydrofuran-2-yl)methyl)amino)-3-nitrophenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-((((trans or cis)-4-hydroxytetrahydrofuran-2-yl)methyl)amino)-3-nitrophenyl)sulfonyl)benzamide;
or a pharmaceutically acceptable salt thereof, or a stereoisomer thereof.

In some embodiments, the Bcl-2 inhibitor in present disclosure is 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide (Compound A) or a pharmaceutically acceptable salt thereof.

### Preparation of Bcl-2 Inhibitors

All the Bcl-2 inhibitors having Formulas (III-B), (III-C), (III-D) or (III-E), including 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide (Compound A), can be prepared by the method disclosed in international publication WO2019/210828A1.

### Preparation of Compound A

Step 1: 2,2-dimethoxy-7-azaspiro[3.5]nonane hydrochloride

To the solution of tert-butyl 2-oxo-7-azaspiro[3.5]nonane-7-carboxylate (500 g, 2.09 mol) in MeOH (750 mL) and EA (750 mL) was added conc. HCl acid (350 mL, 4.18 mol) at room temperature and stirred for 4 hours. After concentrated in vacuum, MeOH (750 mL) was added into the residue and then the resulting mixture was concentrated in vacuum (repeated this work-up twice). The brown residue was suspended in EA (1250 mL) and stirred for 1 hour. The solid precipitation was filtered and dried in vacuum to afford the title product as an off-white powder (350 g, yield: 76.0%). ¹H NMR (400 MHz, DMSO-d₆)δ ppm: 3.03 (s, 6H), 2.96-2.89 (m, 4H), 1.93 (s, 4H), 1.74-1.67 (m, 4H). MS (ESI, m/e) [M+1]⁺ 186.0.

Step 2: methyl 2-((1H-pyrrolo[2,3-b]pyridin-5-yl)oxy)-4-(2,2-dimethoxy-7-azaspiro[3.5]nonan-7-yl)benzoate

The mixture of methyl 2-((1H-pyrrolo[2,3-b]pyridin-5-yl)oxy)-4-fluorobenzoate (100 g), 2,2-dimethoxy-7-azaspiro[3.5]nonane hydrochloride (116 g, 1.5 eq.) and DBU (160 g, 3.0 eq.) in NMP (500 mL) was stirred for 16 hours at 85° C. After the reaction was completed, the mixture was cooled to 50±5° C. and citric acid in water (2%, 5 L) was added drop-wise into the system under stirring. After filtered, the cake was collected and dissolved with DCM (1.5 L). The solution of crude product was washed with citric acid in water (2%, 1.5 L), saturated aq. NaHCO₃ (1.5 L) and 15% aq. NaCl (1.5 L), and then dried over anhydrous Na₂SO₄. Silica gel (100 g) was added into the solution of the crude product under stirring and then filtered. The filtrate was concentrated to 300 mL. MTBE (500 mL) was poured into the system. After stirred for 2 hours, the cake was collected after filtration and was dried in vacuum to give an off-white solid (192 g, yield: 72.1%). ¹H NMR (400 MHz, DMSO-d₆)δ ppm: 11.63 (s, 1H), 8.00 (d, J=2.4 Hz, 1H), 7.76 (d, J=9.2 Hz, 1H), 7.47 (t, J=3.2 Hz, 1H), 7.42 (d, J=2.4 Hz, 1H), 6.79 (dd, J=2.4 Hz, J=9.2 Hz, 1H), 6.39-6.36 (m, 2H), 3.64 (s, 3H), 3.17-3.12 (m, 4H), 3.01 (s, 6H), 1.86 (s, 4H), 1.54-1.50 (m, 4H). MS (ESI, m/e) [M+1]⁺ 451.9.

Step 3: methyl 2-((1H-pyrrolo[2,3-b]pyridin-5-yl)oxy-4-(2-oxo-7-azaspiro[3.5]nonan-7-yl)benzoate

To the solution of methyl 2-((1H-pyrrolo[2,3-b]pyridin-5-yl)oxy)-4-(2,2-dimethoxy-7-azaspiro[3.5]nonan-7-yl)benzoate (176 g, 0.39 mol) in DCM (2 L) was added diluted HCl acid (1M, 1.5 L) and stirred for overnight. After the reaction was completed, the mixture was cooled to 10° C. and was adjusted to pH=8-9 with aqueous NaOH solution (4 M) under stirring. The organic phase was separated and washed with 15% aq. NaCl (1 L), then washed with H₂O (1 L). After the organic phase was concentrated to 500 mL, MTBE (1 L) was poured into the solution and then the system was concentrated to 500 mL (repeated this work-up 3 times). The resulting system was stirred for 0.5 hour. After filtration, the cake was collected and then dried in vacuum to obtain the tittle product as a white solid (152 g, yield: 96.23%). ¹H NMR (400 MHz, DMSO-d₆)δ ppm: 11.64 (s, 1H), 8.02 (d, J=2.4 Hz, 1H), 7.78 (d, J=9.2 Hz, 1H), 7.47 (t, J=3.2 Hz, 1H), 7.44 (d, J=2.4 Hz, 1H), 6.83 (dd, J=2.4 Hz, J=9.2 Hz, 1H), 6.43 (d, J=2.4 Hz, 1H), 6.38-6.36 (m, 1H), 3.65 (s, 3H), 3.24-3.21 (m, 4H), 2.80 (s, 4H), 1.70-1.67 (m, 4H). MS (ESI, m/e) [M+1]⁺ 405.9.

Step 4: (S)-tert-butyl 2-(2-(prop-1-en-2-vlphenvl)pyrrolidine-1-carboxylate

To a mixture of (S)-tert-butyl 2-(2-bromophenyl)pyrrolidine-1-carboxylate (50 g, 153.3 mmol) and 4,4,5,5-tetramethyl-2-(prop-1-en-2-yl)-1,3,2-dioxaborolane (38.6 g, 229.9 mmol) in dioxane (500 mL) and H₂O (50 mL) was added Cs₂CO₃ (100 g, 305 mmol) and Pd(dppf)Cl₂ (6.6 g, 7.5 mmol). The mixture was stirred at 100° C. for 8 hours. TLC showed the reaction was completed. The mixture was concentrated in vacuum. The residue was purified by column chromatography on silica gel (eluent: PE/EA (v/v)=100/1 to 10/1) to obtain (S)-tert-butyl 2-(2-(prop-1-en-2-yl)phenyl)pyrrolidine-1-carboxylate (65 g, crude). The crude product was used directly in next step.

Step 5: (S)-tert-butyl 2-(2-isopropylphenyl)pyrrolidine-1-carboxylate

To a solution of (S)-tert-butyl 2-(2-(prop-1-en-2-yl)phenyl)pyrrolidine-1-carboxylate (30 g, 104.39 mmol) in MeOH (500 mL) was added Pd/C (10 g, 10%) and the mixture was stirred at 20° C. under H₂(15 Psi) for 12 hours. TLC showed the reaction was completed. The mixture was filtered and the filtrate was concentrated in vacuum to give (S)-tert-butyl 2-(2-isopropylphenyl)pyrrolidine-1-carboxylate (60 g, crude), which was used in next step without further purification. ¹H NMR (400 MHz, CDCl₃)δ ppm: 7.39-6.90 (m, 4H), 5.36-5.04 (m, 1H), 3.77-3.52 (m, 2H), 3.20-3.17 (m, 1H), 2.47-2.24 (m, 1H), 1.96-1.65 (m, 3H), 1.54-1.38 (m, 2H), 1.31-1.22 (m, 8H), 1.17 (s, 7H).

Step 6: (S)-2-(2-isopropylphenyl)pyrrolidine hydrochloride

To a solution of tert-butyl 2-(2-isopropylphenyl)pyrrolidine-1-carboxylate (55 g, 190 mmol) in DCM (50 mL) was added HCl in 1,4-dioxane (4 M, 142 mL, 570 mmol) dropwise at room temperature. The mixture was stirred at room temperature for overnight. The mixture was concentrated in vacuum. The resulting residue was slurried with EA (100 mL) and then filtered, dried in vacuum to give (S)-2-(2-isopropylphenyl)pyrrolidine hydrochloride 26 g (yield: 60.4%). ¹H NMR (400 MHz, DMSO-d₆) δ ppm: 9.93 (s, 1H), 8.81 (s, 1H), 7.63-7.57 (m, 1H), 7.41-7.34 (m, 2H), 7.32-7.24 (m, 1H), 4.91-4.75 (m, 1H), 3.47-3.35 (m, 1H), 3.31-3.25 (m, 1H), 2.40-2.21 (m, 1H), 2.19-1.86 (m, 3H), 1.25 (d, J=6.7 Hz, 3H), 1.17 (d, J=6.7 Hz, 3H). MS (ESI, m/e) [M+1]⁺ 190.0.

Step 7: methyl (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoate

A mixture of (S)-2-(2-isopropylphenyl)pyrrolidine hydrochloride (120 g, 0.535 mole) and methyl 2-((1H-pyrrolo[2,3-b]pyridin-5-yl)oxy)-4-(2-oxo-7-azaspiro[3.5]nonan-7-yl)benzoate (218 g, 0.509 mole) in DCM (2.2 L) was charged into a reactor. The temperature was controlled blow 30° C. and NaBH(OAc)₃ (216 g, 1.018 mole) was added into the reactor in 5-6 portions. Then the reaction mixture was stirred at room temperature and monitored by TLC. After the starting material ketone was consumed completely, the mixture was adjusted to pH=4-5 with diluted HCl acid (0.5 M). The separated organic phase was washed with H₂O (600 mL×2) and then washed with aq. NaHCO₃ (600 mL×2), saturated aq. NaCl (600 mL). The organic phase was collected, then dried over anhydrous Na₂SO₄ and concentrated. 256 g off-white solid was obtained as the crude product, which was used in the next step directly. MS (ESI, m/e) [M+1]⁺ 579.0.

Step 8: (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid

To a solution of methyl (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoate (105 g, 181.7 mmol) in THF (525 mL) and MeOH (525 mL) was added aq. NaOH (3.5 M). It was stirred at room temperature overnight. After THF and MeOH were removed in vacuum, 3.5 L of water was added into the residue. The resulting mixture was adjusted to pH=5˜6 with 3 N HCl acid at room temperature with stirring. The precipitate was filtered and dried in vacuum to give the product as a white solid (102.4 g, yield: 99%). ¹H NMR (400 MHz, DMSO-d₆) δ ppm: 12.13 (s, 1H), 11.58 (s, 1H), 7.95 (s, 1H), 7.67 (d, J=8.0 Hz, 1H), 7.56-7.40 (m, 2H), 7.35 (s, 1H), 7.27-7.04 (m, 3H), 6.68 (d, J=8.0 Hz, 1H), 6.32 (s, 2H), 3.62 (s, 1H), 3.32-3.26 (m, 1H), 3.10-3.04 (m, 4H), 2.35-2.30 (m, 1H), 2.9-2.15 (m, 1H), 1.74-1.64 (m, 4H), 1.52-1.37 (m, 6H), 1.28-1.06 (m, 6H). MS (ESI, m/e) [M+1]⁺ 564.9.

Step 9: 2-((1H-pyrrolo[2,3-b]pyridin-5-yl)oxy)-N-((4-((((1r,4r-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide

A mixture of (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid (44 g, 78 mmol), 4-((((1r,4r)-4-hydroxy-4-methylcyclohexyl)methyl)amino)-3-nitrobenzenesulfonamide (26.8 g, 78 mmol), TEA (15.7 g, 156 mmol), EDCI (19.4 g, 101 mmol) and DMAP (19 g, 156 mmol) in anhydrous DCM (880 mL) was stirred overnight at room temperature. The reaction was monitored by HPLC. After starting material of (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid was consumed completely, the reaction mixture was heated to −35° C. and N¹,N¹-dimethylethane-1,2-diamine (17.2 g, 195 mmol) was added in one portion. The reaction was stirred for another 12 hours. The mixture was washed twice with 10 wt % aq. AcOH solution (300 mL×2) and then washed with saturated aq. NaHCO₃ (300 mL×2). The organic layer was collected and concentrated to about 90 mL. 22 g of silica gel was added and stirred for 2 hours. After filtration, 180 mL EA was added into the filtrate at reflux and further stirred for 5 hours. After the mixture was cooled to room temperature, the precipitate was filtered and then the wet cake was washed twice with EA (180 mL). After drying in vacuum at 80-90° C., the desired compound was obtained (48 g, yield: 69.5%). ¹H NMR (DMSO-d₆) δ ppm: 11.65 (s, 1H), 11.11 (br, 1H), 8.58-8.39 (m, 2H), 8.00 (d, J=2.8 Hz, 1H), 7.74 (d, J=8.8 Hz, 1H), 7.57-7.37 (m, 4H), 7.30-7.10 (m, 3H), 7.00 (d, J=9.2 Hz, 1H), 6.65 (d, J=1.2 Hz, 1H), 6.35 (s, 1H), 6.17 (s, 1H), 4.24 (s, 1H), 3.39-3.20 (m, 5H), 3.04-2.88 (m, 4H), 2.23 (s, 1H), 1.94-1.47 (m, 11H), 1.44-1.26 (m, 7H), 1.19 (d, J=8.0 Hz, 3H), 1.14 (d, J=8.0 Hz, 3H), 1.10 (s, 4H). MS (ESI, m/e) [M+1]⁺ 889.9.

### Methods of Treatment

In one aspect, the present disclosure provides a method of treating cancer. In certain aspects, the method comprises administering to a patient a therapeutically effective amount of Compound A, in combination with a therapeutically effective amount of azacitidine. The cancer is myeloid malignancies, selected from the group consisting of acute myeloid leukemia (AML).

Compound A can be administered by any suitable means, including oral, parenteral, intrapulmonary, and intranasal, and, if desired for local treatment, intralesional administration. Dosing can be by any suitable route. Various dosing schedules including but not limited to single or multiple administrations over various time-points, bolus administration, and pulse infusion are contemplated herein.

Compound A would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. Compound A is optionally formulated with one or more agents currently used to prevent or treat the disorder in question. The effective amount of such other agents depends on the amount of Compound A in the formulation, the type of disorder or treatment, and other factors discussed above.

For the prevention or treatment of disease, the appropriate dosage of Compound A will depend on the type of disease to be treated, the severity and course of the disease, whether Compound A is administered for preventive or therapeutic purposes, previous therapy, the patient's clinical history and response to Compound A, and the discretion of the attending physician. Compound A is suitably administered to the patient at one time or over a series of treatments.

### Examples

The present invention is further exemplified, but not limited to, by the following examples that illustrate the invention.

### Example 1: Clinical Study

### 1. Method

### Study Design/Objectives

The clinical study was conducted to evaluate the combination of Compound A and azacitidine in patients with myeloid malignancies, including acute myeloid leukemia (AML) (either treatment-naïve [TN] unfit for intensive induction chemotherapy or relapsed/refractory [R/R]), myelodysplastic syndrome (MDS), or MDS/myeloproliferative neoplasm (MPN). Patients with AML who were treatment-naive unfit for intensive chemotherapy or who are relapsed/refractory were enrolled into the AML cohorts; and patients with myelodysplastic syndrome (MDS) or MDS/myeloproliferative neoplasm (MPN) who were treatment-naïve with high-risk disease or who were relapsed/refractory were enrolled into the MDS cohorts.

### AML Cohorts:

The AML cohorts enrolls patients with treatment-naive (TN) AML who are unfit for intensive chemotherapy and relapsed/refractory (R/R) AML. The treatment cycles are of 28 days for all AML cohorts, unless recommended otherwise by the Safety Monitoring Committee (SMC).

Part 1 AML (Dose Regimen Finding; N≈18 to 36, i.e., a minimum of 6 and a maximum of 12/cohort) studied the safety and tolerability of different dose regimens of Compound A in combination with a fixed dose of azacitidine at 75 mg/m² for 7 days. After 3 patients have completed the dose-limiting toxicities (DLT) window, the SMC reviewed all available data and considered the probability of DLT rate to be >20% and recommended whether or not to enroll additional patients (ie, up to 12). If no additional patients were required, the SMC would recommend whether or not to open a subsequent dose regimen in Part 1 and whether to open the dose regimen for Part 2 expansion.

Each cohort had a 4-day ramp-up of Compound A in the Cycle 1 only, with 12.5% of the target dose on Day 1, 25% on Day 2, 50% on Day 3, and 100% on Day 4. The window to assess dose-limiting toxicity (DLT) is up to Day 28 for nonhematologic toxicities and Day 42 or initiation of Cycle 2 for hematologic toxicities.

The AML Part 1 dose regimen finding cohorts are as follows:
A) Compound A 40 mg daily×10 days with azacitidine (Ramp-up dosing of Compound A: 5 mg, 10 mg, 20 mg, 40 mg)
B) Compound A 80 mg daily×10 days with azacitidine (Ramp-up dosing of Compound A: 10 mg, 20 mg, 40 mg, 80 mg)
C) Compound A 160 mg daily×10 days with azacitidine (Ramp-up dosing of Compound A: 20 mg, 40 mg, 80 mg, 160 mg)
D) Compound A 160 mg daily×28 days with azacitidine (Ramp-up dosing of Compound A: 20 mg, 40 mg, 80 mg, 160 mg)
E) Compound A 320 mg daily×21 days with azacitidine (Ramp-up dosing of Compound A: 40 mg, 80 mg, 160 mg, and 320 mg)
F) Compound A 320 mg daily×28 days with azacitidine (Ramp-up dosing of Compound A: 40 mg, 80 mg, 160 mg, and 320 mg)

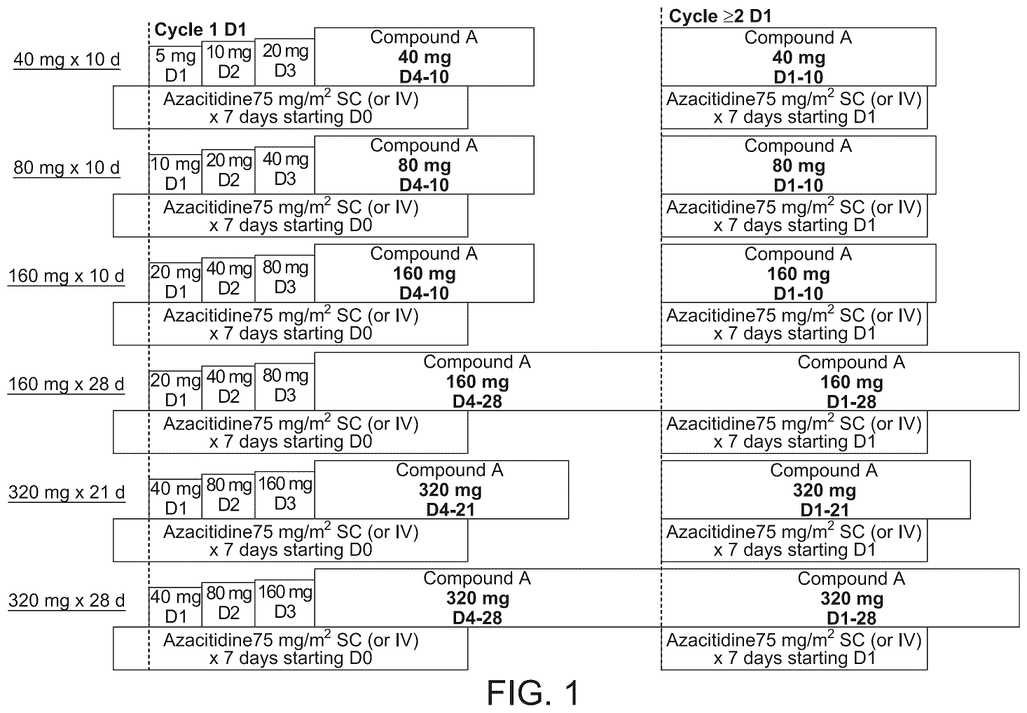

In all Part 1 AML cohorts and the Part 2 cohort receiving the 28-day dose regimen, the initial dose of azacitidine is given 1 day prior to administration of Compound A (designated as Day 0) to compare baseline PK samples of azacitidine monotherapy with azacitidine in combination with Compound A, which starts on Day 1. In the subsequent cycles and all remaining cohorts, azacitidine administration start on Day 1 concurrently with Compound A. Dosing schema in Part I AML is shown in FIG. 1.

Part 2 AML (Safety Expansion; N≈60) treated up to the cohorts each consisting of approximately 10 patients with dose regimens as defined in Part 1 and recommended by the SMC to proceed to Part 2. Part 2 of the study determined safety and antileukemic activity in a larger number of patients with a longer follow-up at dose regimens that were declared tolerable with early signs of activity in Part 1. At a given time, more than one dose regimen may be approved for expansion in Part 2; however, only a single Part 2 AML cohort was to be offered to prospective participants. Patients were preferentially enrolled in any open Part 1 cohort, if applicable. Each Part 2 cohort would continue to enroll up to 10 patients; this would include any additional patients enrolled in Part 1 but not needed for the initial SMC safety review of that cohort. Based upon the SMC's recommendation, a cohort may be closed before enrolling 10 patients or expanded to enroll additional 10 patients to gather additional data at a particular dose regimen. After completion of enrollment in all the Part 2 AML cohorts, the recommended dose would be determined by the SMC based on safety, tolerability, and PK.

The dose regimen of Compound A 160 mg once daily for 10 days with azacitidine 75 mg/m² could be selected as the recommended dose for Part 3 unless another tested dose regimen appears superior based on SMC assessment.

Part 3 AML (Efficacy Expansion; N≈40) treated approximately 20 patients with the recommended dose determined in Part 2 AML cohorts. All AML patients would be enrolled in a single Part 3 AML cohort; however, based on differences in safety and tolerability of AML patients, the AML cohort may be split into 2 separate cohorts: TN AML patients are unfit for intensive chemotherapy and R/R AML patients.

A subset of the Part 3 AML cohort will receive a modified second cycle of treatment to explore DDI with posaconazole, a strong cytochrome P450 (CYP) 3A4 inhibitor. The schedule for the second cycle will depend on whether a 10-day or 28-day regimen is selected as the AML recommended dose. If a 10-day dose regimen is selected, patients will be treated at the recommended dose for the first 10 days of Cycle 2. Patients will then receive Compound A at one-eighth the recommended dose from Day 11 to Day 20. Posaconazole will be given at 300 mg twice daily (BID) on Day13 and then 300 mg once daily from Day 14 to Day 20 with food (30 minutes prior to Compound A administration). If a 28-day Compound A course is chosen, Compound A at one-eighth the recommended dose will be given from Day 11 to Day 28 of Cycle 2 along with posaconazole from Day 13 to Day 20. PK sampling will occur on Days 12 to 13 and Days 20 to 21 of Cycle 2. Normal dosing will resume from Cycle 3 onwards.

AML Monotherapy Part (N≈10) studied the safety and tolerability of Compound A as a monotherapy in patients with R/R. After 3 patients have completed the dose-limiting toxicities (DLT) window, the SMC would review all available data and consider the probability of DLT rate to be >20% and would recommend whether or not to enroll additional patients.

The SMC would also recommend whether the dose level needed to be modified.

The monotherapy cohort had a 4-day ramp-up of Compound A in the first cycle only, with 12.5% of the target dose on Day 1, 25% on Day 2, 50% on Day 3, and 100% on Day 4.

The R/R AML monotherapy cohort is as follows:
Compound A 160 mg daily×28 days (Ramp-up dosing of Compound A: 20 mg, 40 mg, 80 mg, and 160 mg)
The target dose is 160 mg daily×28 days unless another dose level was selected by the SMC as the dose.

The monotherapy cohort received Compound A once daily for 28 days with a target dose of 160 mg (selected by the SMC as the recommended dose).

### Inclusion Criteria

Each patient eligible to participate in this study must meet all the applicable criteria:
1. Age 18 years or older
2. Confirmed diagnosis of one of the following by 2016 World Health Organization criteria:
   a. AML, nonacute promyelocytic leukemia
3. Eastern Cooperative Oncology Group (ECOG) performance status of 0 to 2.
4. Adequate organ function defined as:
   a. Creatinine clearance ≥45 mL/min (or between 30 and 45 mL/min in unfit AML cohort)
   b. Adequate liver function
5. Life expectancy of >12 weeks
6. Ability to comply with the requirements of the study

### Exclusion Criteria

Each patient eligible to participate in this study must not meet any of the following exclusion criteria:
1. A diagnosis of acute promyelocytic leukemia
2. Prior malignancy within the past 2 years, except for curatively treated localized skin cancer, superficial bladder cancer, carcinoma in situ of the cervix or breast, or localized Gleason score ≤6 prostate cancer
3. Antecedent MPN including myelofibrosis, essential thrombocytosis, polycythemia vera, or chronic myelogenous leukemia with or without BCR-ABL1 translocation and AML with BCR-ABL1 translocation
4. Known central nervous system involvement by leukemia
5. Prior therapy with a Bcl-2 inhibitor or azacytidine

### MDS Cohorts:

The MDS cohorts enrolled patients with TN MDS and TN MDS/MPN or R/R MDS and R/R MDS/MPN who had received at least 1 systemic therapy. In the MDS cohorts, treatment cycles was of 28 days, unless modified by the SMC.

Part 1 MDS (Dose Regimen Finding, N≈12 to 24) treated a minimum of 3 and a maximum of 6 MDS patients/cohort each dose regimen of Compound A in combination with azacitidine 75 mg/m² for 7 days.

Each cohort would have a 4-day ramp-up of Compound A in the first cycle only, with 12.5% of the target dose on Day 1, 25% on Day 2, 50% on Day 3, and 100% on Day 4.

The MDS Part 1 dose regimen finding cohorts were as follows:
A) Compound A 40 mg daily×10 days with azacitidine (Ramp-up dosing of Compound A: 5 mg, 10 mg, 20 mg, and 40 mg)
B) Compound A 80 mg daily×10 days with azacitidine (Ramp-up dosing of Compound A: 10 mg, 20 mg, 40 mg, and 80 mg)
C) Compound A 160 mg daily×10 days with azacitidine (Ramp-up dosing of Compound A: 20 mg, 40 mg, 80 mg, and 160 mg)
D) Compound A 160 mg daily×21 days with azacitidine (Ramp-up dosing of Compound A: 20 mg, 40 mg, 80 mg, and 160 mg)

Compound A once daily for 10 days with a target dose of 160 mg in combination with azacitidine. This cohort would open after the safety data from the first 2 cohorts of AML patients enrolled in Part 1 was reviewed by the SMC and no safety concerns were identified. Compound A in Cycle 1 would be given with a 4-day ramp-up: 20 mg, 40 mg, 80 mg, 160 mg. It is anticipated that a single dose regimen will be tested in Part 1 MDS, although the SMC may elect to test another dose regimen following review of data from this or subsequent MDS or AML cohorts. After 3 patients in a cohort have completed one cycle of treatment, the SMC would review all available data before opening the subsequent cohort, if any. The SMC may recommend treating additional patients in a cohort before confirming tolerability.

Part 2 MDS (Safety Expansion, N≈40), treated up to 10 patients at dose regimen(s) selected in Part 1 MDS. At any given time, a single Part 2 MDS cohort would be open to enrollment. After completion of enrollment in all the Part 2 MDS cohorts, the recommended dose would be determined by the SMC based on safety, tolerability, and PK data from these Part 2 cohorts and any available activity and/or exploratory data.

The dose regimen of Compound A 160 mg once daily for 10 days with azacitidine 75 mg/m² was selected as the recommended dose for Part 3.

Part 3 MDS (Efficacy Expansion, N≈40) treated approximately 20 patients at the MDS dose determined in Part 2 MDS. Enrollment to Part 3 would be opened after Part 2 cohorts have completed enrollment and the SMC recommendation was obtained after the review of all available data.

All Part 1, Part 2, and Part 3 cohorts were assessed for DLTs. The DLT window will start with the first dose of study drug and last until Day 28 for nonhematologic toxicities, and until Day 42 of Cycle 1 or the initiation of Cycle 2 for hematologic toxicities.

MDS Monotherapy Part (N≤10) studied the safety and tolerability of Compound A as monotherapy in patients with MDS or MDS/MPN. After 3 patients have completed the DLT window, the SMC would review all available data and consider the probability of DLT rate to be >20% and would recommend whether or not to enroll additional patients. The SMC would also recommend whether the dose level needs to be modified.

The monotherapy cohort would have a 4-day ramp-up of Compound A in the first cycle only, with 12.5% of the target dose on Day 1, 25% on Day 2, 50% on Day 3, and 100% on Day 4.

The MDS, MDS/MPN monotherapy cohort is as follows:
Compound A 160 mg daily×28 days (Ramp-up dosing of Compound A: 20 mg, 40 mg, 80 mg, and 160 mg)
The target dose is 160 mg daily×28 days unless another dose level is selected by the SMC as the dose.

The dose regimen of Compound 160 mg once daily for 10 days with azacitidine 75 mg/m² could be selected as the recommended dose for Part 3 unless another dose regimen was considered likely to be superior in terms of toxicity or efficacy.

### 2. Results and Conclusion

### Safety

As of 4 Feb. 2023, 97 patients have been enrolled, including 66 with AML and 31 with MDS or MDS/MPN. Evaluation of Part 1 AML cohorts for the 10-day regimen of Compound A at 40, 80, and 160 mg, and the 28-day regimen of 160 mg in combination with azacitidine have been completed by the SMC and respective Part 2 expansion cohorts have also been opened for enrollment. The 21-day regimen of Compound A at 320 mg has also opened for enrollment for Part 1.

In MDS or MDS/MPN, evaluation of Part 1 cohorts for the 10-day regimen of Compound A at 40, 80, and 160 mg in combination with azacitidine have been completed by the SMC and respective Part 2 expansion cohorts have also been opened for enrollment.

The safety analysis showed that the use of Compound A in combination with azacitidine in the treatment of AML or MDS or MDS/MPN was well-tolerated and safe.

### Efficacy

At the time of data cutoff (5 Sep. 2022), 57 patients with AML (31 TN and 26 R/R AML) had been treated with azacitidine in combination with Compound A (40 mg×10 days [n=16]; 80 mg×10 days [n=17], 160 mg×10 days [n=16], and 160 mg×28 days [n=8]). In the overall population, the median duration of treatment was 3 months (range: 0.1-15.4). Twenty-four (42%) patients had adverse cytogenetics and 23 (40%) had bone marrow blasts ≥50% at study entry.

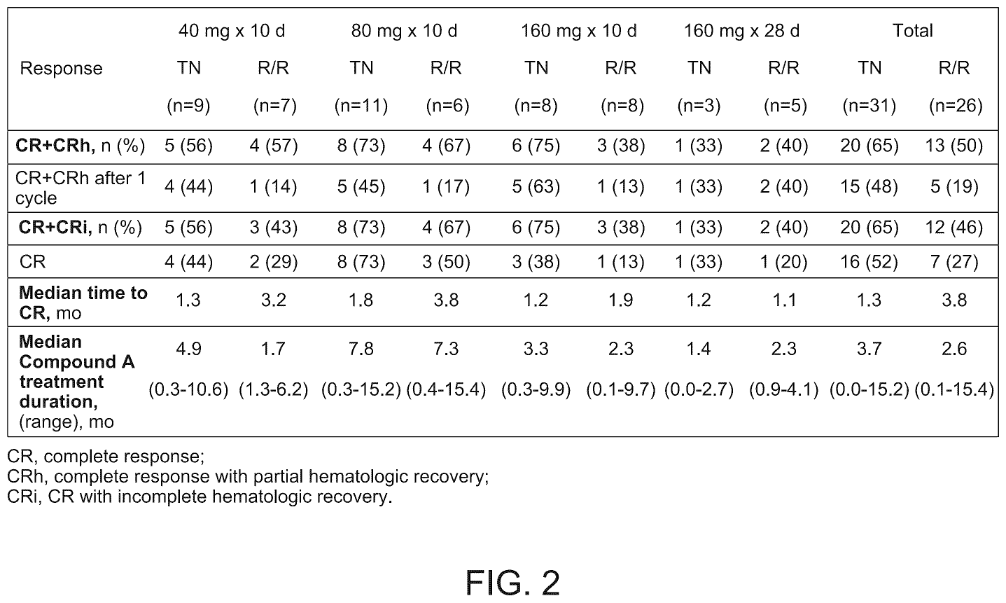

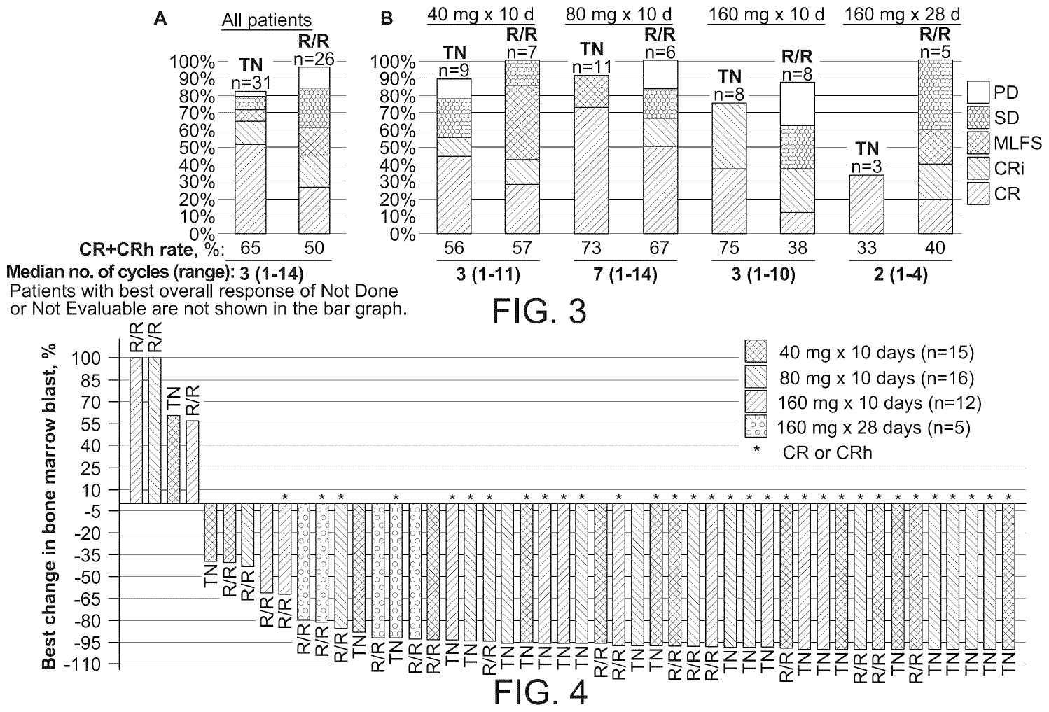

CR/CRh was achieved in 65% of TN unfit AML and 50% in patients with R/R AML; Most CR+CRh in TN AML (15 of 20) was achieved by the end of cycle 1. FIG. 2 is a summary of Complete Responses in Patients with AML. The 80 mg×10 day cohort (n=17) had the longest treatment duration with a median of 7 cycles (FIG. 3); CR+CRh was seen in 73% and 67% of TN and R/R patients, respectively; and CR was seen in 73% and 50% of TN and R/R patients, respectively. Reduction in bone marrow blast is shown in FIG. 4.

The foregoing examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entireties.

### REFERENCES

Adams, J. M., and Cory, S. (2007) The Bcl-2 apoptotic switch in cancer development and therapy. Oncogene 26, 1324-1337
Anderson, M. A., Huang, D., and Roberts, A. (2014) Targeting BCL2 for the treatment of lymphoid malignancies. Semin Hematol 51, 219-227
Czabotar, P. E., Lessene, G., Strasser, A., and Adams, J. M. (2014) Control of apoptosis by the BCL-2 protein family: implications for physiology and therapy. Nat Rev Mol Cell Biol 15, 49-63
Egle, A., Harris, A. W., Bath, M. L., O'Reilly, L., and Cory, S. (2004) VavP-Bcl2 transgenic mice develop follicular lymphoma preceded by germinal center hyperplasia. Blood 103, 2276-2283
Kondo, S., Oakes, M. G., and Sorenson, C. M. (2008) Rescue of renal hypoplasia and cystic dysplasia in Bcl-2 −/− mice expressing Bcl-2 in ureteric bud derived epithelia. Dev Dyn 237, 2450-2459
Roberts, A. W. (2016) Targeting apoptotic pathways to treat lymphoid malignancies. Rinsho Ketsueki 57, 2054-2058
Roberts, A. W., and Huang, D. (2017) Targeting BCL2 With BH3 Mimetics: Basic Science and Clinical Application of Venetoclax in Chronic Lymphocytic Leukemia and Related B Cell Malignancies. Clin Pharmacol Ther 101, 89-98
Schenk, R. L., Strasser, A., and Dewson, G. (2017) BCL-2: Long and winding path from discovery to therapeutic target. Biochem Biophys Res Commun 482, 459-469
Tausch, E., Close, W., Dolnik, A., Bloehdorn, J., Chyla, B., Bullinger, L., Dohner, H., Mertens, D., and Stilgenbauer, S. (2019) Venetoclax resistance and acquired BCL2 mutations in chronic lymphocytic leukemia. Haematologica 104, e434-e437.
Veis, D. J., Sorenson, C. M., Shutter, J. R. and Korsmeyer, S. J. (1993) Bcl-2-deficient mice demonstrate fulminant lymphoid apoptosis, polycystic kidneys, and hypopigmented hair. Cell, 75, 229-240.
Yamamura, K., Kamada, S., Ito, S., Nakagawa, K., Ichihashi, M., and Tsujimoto, Y. (1996) Accelerated disappearance of melanocytes in bcl-2-deficient mice. Cancer Res 56, 3546-3550.

1. A method of treating myeloid malignancies in a subject, the method comprising administering to the subject a therapeutically effective amount of a Bcl-2 inhibitor, in combination with a therapeutically effective amount of azacitidine, wherein the myeloid malignancies are acute myeloid leukemia (AML), myelodysplastic syndrome (MDS), or MDS/myeloproliferative neoplasm (MPN); and
the Bcl-2 inhibitor is selected from the group consist of:
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(((4-fluorotetrahydro-2H-pyran-4-yl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-ethylphenyl)pyrrolidin-1-yl)-7-azaspiro[3.5]nonan-7-yl)-N-((4-(((4-fluorotetrahydro-2H-pyran-4-yl)methyl)amino)-3-nitrophenyl)sulfonyl)benzamide;
2-((1H-pyrrolo[2,3-b]pyridin-5-yl)oxy)-4-(2-((S)-2-(2-ethylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
(R)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(((4-fluorotetrahydro-2H-pyran-4-yl)methyl)amino)-3-nitrophenyl)sulfonyl)benzamide;
2-((1H-pyrrolo[2,3-b]pyridin-5-yl)oxy)-4-(2-((R)-2-(2-cyclopropylphenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
(S)-2-((1H-pyrrolo[2,3-b]pyridin-5-yl)oxy)-4-(7-(2-(2-cyclopropylphenyl)pyrrolidin-1-yl)-2-azaspiro[3.5]nonan-2-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(7-(2-(2-cyclopropylphenyl)pyrrolidin-1-yl)-2-azaspiro[3.5]nonan-2-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(9-(2-(2-cyclopropylphenyl)pyrrolidin-1-yl)-3-azaspiro[5.5]undecan-3-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(9-(2-(2-cyclopropylphenyl)pyrrolidin-1-yl)-3-azaspiro[5.5]undecan-3-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(6-(2-(2-cyclopropylphenyl)pyrrolidin-1-yl)-2-azaspiro[3.3]heptan-2-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(6-(2-(2-cyclopropylphenyl)pyrrolidin-1-yl)-2-azaspiro[3.3]heptan-2-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(3-chloro-2-(dimethylamino)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)benzamide;
(S)2-((1H-pyrrolo[2,3-b]pyridin-5-yl)oxy)-4-(2-((S)-2-(2-cyclopropylphenyl)pyrrolidin-1-yl)-8-azaspiro[4.5]decan-8-yl)-N-((3-nitro-4-(((tetrahydro-2H-pyran-4-yl)methyl)amino)phenyl)sulfonyl)benzamide;
(R) 2-((1H-pyrrolo[2,3-b]pyridin-5-yl)oxy)-4-(2-((S)-2-(2-cyclopropylphenyl)pyrrolidin-1-yl)-8-azaspiro[4.5]decan-8-yl)-N-((3-nitro-4-(((tetrahydro-2H-pyran-4-yl)methyl)amino)phenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-ethylphenyl)pyrrolidin-1-yl)-7-azaspiro[3.5]nonan-7-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-ethylphenyl)pyrrolidin-1-yl)-7-azaspiro[3.5]nonan-7-yl)benzamide;
(S)-2-((1H-pyrrolo[2,3-b]pyridin-5-yl)oxy)-4-(8-(2-(2-cyclopropylphenyl)pyrrolidin-1-yl)-2-azaspiro[4.5]decan-2-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-N-((4-(((4-fluorotetrahydro-2H-pyran-4-yl)methyl)amino)-3-nitrophenyl)sulfonyl)-4-(2-(2-(2-isopropylphenyl)pyrrolidin-1-yl)-7-azaspiro[3.5]nonan-7-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1s,4s)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((R)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1s,4s)-4-hydroxy-4-methylcyclohexyl)methyl)amino)-3-nitrophenyl)sulfonyl)-4-(2-((R)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-4-(2-(2-(2-cyclobutylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-isobutylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(3-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-N-((3-nitro-4-(((tetrahydro-2H-pyran-4-yl)methyl)amino)phenyl)sulfonyl)-4-(2-(2-(o-tolyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-bromophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(2-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(3-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(3-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(4-chlorophenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-ethoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-(dimethylamino)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-((S)-2-(2-(dimethylamino)phenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(2-((S)-2-(2-(bis(methyl-d3)amino)phenyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-N-((3-nitro-4-(((tetrahydro-2H-pyran-4-yl)methyl)amino)phenyl)sulfonyl)-4-(2-(2-(2-(pyrrolidin-1-yl)phenyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-(1-methyl-1,2,3,6-tetrahydropyridin-4-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-(1-methylpiperidin-4-yl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-methoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(2-isopropoxyphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-(methoxymethyl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-(hydroxymethyl)phenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(3-chloro-2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S)-2-((1H-pyrrolo[2,3-b]pyridin-5-yl)oxy)-4-(2-(2-(3-chloro-2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(5-chloro-2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(R)-2-((1H-pyrrolo[2,3-b]pyridin-5-yl)oxy)-4-(2-(2-(3-chloro-2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
(S or R)-2-((1H-pyrrolo[2,3-b]pyridin-5-yl)oxy)-4-(2-(2-(3-chloro-2-ethylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2,4-dicyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2,5-dicyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(3-(2-chlorophenyl)thiophen-2-yl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-methylpyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(4-cyclopropyl-2-(2-cyclopropylphenyl)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-phenyl-2,5-dihydro-1H-pyrrol-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4,4-dimethylpyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4,4-difluoropyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-(trifluoromethyl)pyrrolidin-1-yl)-7-azaspiro[3.5]nonan-7-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(2-(2-(2-cyclopropylphenyl)-4-(dimethylamino)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-4-(2-(dimethylamino)ethoxy)pyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-(2-(2-cyclopropylphenyl)-3,3-dimethylpyrrolidin-1-yl)-7-azaspiro[3.5]nonan-7-yl)-N-((3-nitro-4-(((tetrahydro-2H-pyran-4-yl)methyl)amino)phenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-(((((1s,4s) or (1r,4r))-4-((dimethyl(oxo)-16-sulfaneylidene)amino)cyclohexyl)methyl)amino)-3-nitrophenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-(methyl(3-nitro-4-(((tetrahydro-2H-pyran-4-yl)methyl)amino)phenyl)(oxo)-16-sulfaneylidene)benzamide;
2-((1H-pyrrolo[2,3-b]pyridin-5-yl)oxy)-N-((4-(((-3-oxabicyclo[3.1.0]hexan-6-yl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(((4-hydroxy-4-(trifluoromethyl)cyclohexyl)methyl)amino)-3-nitrophenyl)sulfonyl)benzamide;
2-((1H-pyrrolo[2,3-b]pyridin-5-yl)oxy)-N-((4-((((1r,4r)-4-hydroxy-4-(trifluoromethyl)cyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-N-((4-((((1s,4s)-4-hydroxy-4-(trifluoromethyl)cyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide;
2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)-N-((4-((((1r,4r)-4-methoxy-4-methylcyclohexyl)methyl)amino)-3-nitrophenyl)sulfonyl)benzamide;
2-((1H-pyrrolo[2,3-b]pyridin-5-yl)oxy)-4-(2-((S)-2-(2-isopropylphenyl)pyrrolidin-1-yl)-7-azaspiro[3.5]nonan-7-yl)-N-((4-((((S)-4-methylcyclohex-3-en-1-yl)methyl)amino)-3-nitrophenyl)sulfonyl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-(prop-1-en-2-yl)phenyl)pyrrolidin-1-yl)-7-azaspiro[3.5]nonan-7-yl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-propylphenyl)pyrrolidin-1-yl)-7-azaspiro[3.5]nonan-7-yl)benzamide;
2-((1H-pyrrolo[2,3-b]pyridin-5-yl)oxy)-4-(6-((S)-2-(2-cyclopropylphenyl)pyrrolidin-1-yl)-2-azaspiro[3.3]heptan-2-yl)-N-((4-((((1r,4r)-4-hydroxy-4-methylcyclohexyl)methyl)amino)-3-nitrophenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-cyclopropylphenyl)pyrrolidin-1-yl)-2-azaspiro[3.3]heptan-2-yl)benzamide;
N-((4-((((R)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-cyclopropylphenyl)pyrrolidin-1-yl)-2-azaspiro[3.3]heptan-2-yl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(6-((S)-2-(2-isopropylphenyl)pyrrolidin-1-yl)-2-azaspiro[3.3]heptan-2-yl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-isopropylphenyl)pyrrolidin-yl)-2-azaspiro[3.3]heptan-2-yl)benzamide;
2-((1H-pyrrolo[2,3-b]pyridin-5-yl)oxy)-4-(6-((S)-2-(2-ethylphenyl)pyrrolidin-1-yl)-2-azaspiro[3.3]heptan-2-yl)-N-((4-((((1r,4r)-4-hydroxy-4-methylcyclohexyl)methyl)amino)-3-nitrophenyl)sulfonyl)benzamide;
N-((4-((((S)-1,4-dioxan-2-yl)methyl)amino)-3-nitrophenyl)sulfonyl)-2-((1H-pyrrolo[2,3-b]pyridin-5-yl)oxy)-4-(6-((S)-2-(2-ethylphenyl)pyrrolidin-1-yl)-2-azaspiro[3.3]heptan-2-yl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-((2-(tetrahydro-2H-pyran-4-yl)ethyl)amino)phenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-((2-morpholinoethyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-((2-(3-oxomorpholino)ethyl)amino)phenyl)sulfonyl)benzamide;
2-((1H-pyrrolo[2,3-b]pyridin-5-yl)oxy)-N-((4-(((3-oxabicyclo[3.1.0]hexan-6-yl)methyl)amino)-3-nitrophenyl)sulfonyl)-4-(2-((S)-2-(2-cyclopropylphenyl)pyrrolidin-1-yl)-7-azaspiro[3.5]nonan-7-yl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-(((2,6-dimethyltetrahydro-2H-pyran-4-yl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((3-nitro-4-(((2,2,6,6-tetramethyltetrahydro-2H-pyran-4-yl)methyl)amino)phenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-6-azaspiro[3.4]octan-6-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(6-((S)-2-(2-cyclopropylphenyl)pyrrolidin-1-yl)-2-azaspiro[3.4]octan-2-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-((7R or 7S)-7-((S)-2-(2-cyclopropylphenyl)pyrrolidin-1-yl)-2-azaspiro[4.4]nonan-2-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-((7S or 7R)-7-((S)-2-(2-cyclopropylphenyl)pyrrolidin-1-yl)-2-azaspiro[4.4]nonan-2-yl)-N-((4-((((1r,4r)-4-hydroxy-4-methylcyclohexyl)methyl)amino)-3-nitrophenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-(((2,2-dimethyltetrahydro-2H-pyran-4-yl)methyl)amino)-3-nitrophenyl)sulfonyl)benzamide;
(S)-2-((1H-pyrrolo[2,3-b]pyridin-5-yl)oxy)-4-(2-(2-(2-cyclopropylphenyl)pyrrolidin-1-yl)-7-azaspiro[3.5]nonan-7-yl)-N-((4-(3-methyl-3-((tetrahydro-2H-pyran-4-yl)methyl)ureido)-3-nitrophenyl)sulfonyl)benzamide;
2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-phenylpyrrolidin-1-yl)-7-azaspiro[3.5]nonan-7-yl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-((((cis or trans)-4-hydroxytetrahydrofuran-2-yl)methyl)amino)-3-nitrophenyl)sulfonyl)benzamide;
2-((1H-pyrrolo[2,3-b]pyridin-5-yl)oxy)-4-(2-((S)-2-(2-cyclopropylphenyl)pyrrolidin-1-yl)-7-azaspiro[3.5]nonan-7-yl)-N-((4-((((trans or cis)-4-hydroxytetrahydrofuran-2-yl)methyl)amino)-3-nitrophenyl)sulfonyl)benzamide;
or a pharmaceutically acceptable salt thereof, or a stereoisomer thereof.

2. The mothed of claim 1, wherein the Bcl-2 inhibitor is 2-((1H-pyrrolo[2,3-b]pyridin-5-yl)oxy)-N-((4-((((1r,4r)-4-hydroxy-4-methylcyclohexyl)methyl)amino)-3-nitrophenyl)sulfonyl)-4-(2-((S)-2-(2-isopropylphenyl)pyrrolidin-1-yl)-7-azaspiro[3.5]nonan-7-yl)benzamide or a pharmaceutically acceptable salt thereof.

3. The method of claims 1 or 2, wherein the acute myeloid leukemia (AML) is either treatment-naïve (TN) unfit for intensive induction chemotherapy or relapsed/refractory (R/R).

4. The method of claims 1 or 2 comprising orally administering once daily the Bcl-2 inhibitor to the patient in escalating doses, wherein the escalating doses comprise an initial dose of 5, 10, 20, or 40 mg of the Bcl-2 inhibitor per day.

5. The method of claim 4, wherein the Bcl-2 inhibitor is orally administrated in two Cycles, comprising a 4-day ramp-up schedule (Cycle 1) and a subsequent cycle (Cycle 2).

6. The method of claim 5, wherein each of the 4-day ramp-up schedule (Cycle 1) is a 10-day cycle, 21-day cycle, or a 28-day cycle; and the subsequent cycles (Cycle 2) is a 10-day cycle, 21-day cycle, or a 28-day cycle.

7. The method of claim 6, wherein the 4-day ramp-up schedule (Cycle 1) of the Bcl-2 inhibitor administration comprises the first dose at Day 1, the second dose on Day 2, the third dose on Day 3, and a recommended dose on Day 4 and beyond, wherein the recommended dose on Day 4 and beyond is higher than the second dose on Day 3, the third dose on Day 3 is higher than the second dose on Day 2, and the second dose on Day 2 is higher than the second dose on Day 1.

8. The method of claim 7, wherein the 4-day ramp-up schedule (Cycle 1) of the Bcl-2 inhibitor administration comprises the first dose on Day 1 at 12.5% of the recommended dose, the second dose on Day 2 at 25% of recommended dose, the third dose on Day 3 at 50% of recommended dose, and the daily dose on Day 4 and beyond at 100% of the recommended dose.

9. The method of claim 8, wherein subsequent cycle (Cycle 2) of the Bcl-2 inhibitor administration is at a dose of a recommended dose from Day 1 with no ramp-up.

10. The method of claim 9, wherein azacitidine in Cycle 1 is administrated 1 day prior to the administration of the Bcl-2 inhibitor (designated as Day 0).

11. The method of claim 10, wherein azacitidine in Cycle 2 is administrated concurrently with the Bcl-2 inhibitor.

12. The method of claim 11, wherein azacitidine is administrated at a dose of 75 mg/m2 intravenously or subcutaneously once daily (QD).

13. The method of claim 12, wherein the recommended dose of the Bcl-2 inhibitor is 160 mg once daily (QD).
